(19) United States
(12) Patent Application Publication
(10) Pub. No.: US 2022/0162714 A1
(43) Pub. Date: May 26, 2022

(54) METHOD, SYSTEM AND APPARATUS FOR DETECTION

(71) Applicant: Tangen Bioscience Inc., Branford, CT (US)

(72) Inventors: John F. Davidson, Guilford, CT (US); Arnau Casanovas-Massana, New Haven, CT (US); Zheng Xue, Madison, CT (US); Tony Joaquim, Milford, CT (US)

(73) Assignee: Tangen Bioscience Inc., Branford, CT (US)

(21) Appl. No.: 17/400,136

(22) Filed: Aug. 12, 2021

Related U.S. Application Data
(60) Provisional application No. 63/117,446, filed on Nov. 23, 2020.
(60) Provisional application No. 63/117,434, filed on Nov. 23, 2020.
(60) Provisional application No. 63/117,442, filed on Nov. 23, 2020.

(51) Int. Cl. C12Q 1/70; C12Q 1/6806; C12Q 1/6895

(52) U.S. Cl. CPC C12Q 1/701; C12Q 1/6895; C12Q 1/6806

(57) ABSTRACT

The use of Nucleic Acid Amplification Technologies (NAATs) to rapidly copy a specific fragment of DNA from a few starting molecules has been used to determine the presence of that DNA in a sample. It is of importance for various applications including the identification of a pathogen in a clinical sample. The disclosed embodiments describe an apparatus, disc, methods, and a system for detecting microorganisms such as pathogenic viruses and bacterial rapidly.

### RELATED APPLICATIONS

This application is related to U.S. Provisional Application having U.S. Ser. No. 63/117,446 filed Nov. 23, 2020, by John Davidson, entitled “Method, System and Apparatus for detection”; U.S. Provisional Application U.S. Ser. No. 63/117,434, filed Nov. 23, 2020, by John Davidson, entitled ‘Method, System, and Apparatus for Blood Processing Unit’; and U.S. Provisional Application U.S. Ser. No. 63/117,442, filed Nov. 23, 2020, by John Davidson, entitled Method, System, and Apparatus for Respiratory Testing’, all incorporated by reference herein.

### FIELD

This invention relates generally to nucleic acid amplification, and more particularly to methods, compositions, systems and technologies for amplification of nucleic acids for the detection of particular microorganisms such as viruses and bacteria in a mammal.

### SEQUENCE LISTING

The instant application contains a Sequence Listing which has been submitted electronically in ASCII format and is hereby incorporated by reference in its entirety. Said ASCII copy, created on Nov. 18, 2021, is named TNG-1100-US_SL.txt and is 4,405 bytes in size.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

Nucleic acid analysis methods based on the complementarity of nucleic acid nucleotide sequences can analyze genetic traits directly. Thus, these methods are a very powerful means for identification of genetic diseases, cancer, microorganisms etc. Nucleic acid amplification technologies (NAAT) allow detection and quantification of a nucleic acid in a sample with high sensitivity and specificity. NAAT techniques may be used to determine the presence of a particular template nucleic acid in a sample, as indicated by the presence of an amplification product (i.e., amplicon) following the implementation of a particular NAAT. Conversely, the absence of any amplification product indicates the absence of template nucleic acid in the sample. Such techniques are of great importance in diagnostic applications, for example, for determining whether a pathogen is present in a sample. Thus, NAAT techniques are useful for detection and quantification of specific nucleic acids for diagnosis of infectious and genetic diseases.

Identification of pathogens via direct detection of specific and unique DNA or RNA sequences has been exploited for clinical diagnostic purposes for some time. Molecular detection technologies typically have high analytical sensitivity and specificity compared to antigen and antibody-based methods. Detection of specific genomic DNA or RNA is achieved via amplification of small unique regions of the genome via NAATs such as polymerase chain reaction (PCR, RT-PCR) as well as isothermal methods including loop mediated isothermal amplification (LAMP, RT-LAMP), nucleic acid sequence-based amplification (NASBA), nicking enzyme amplification reaction (NEAR) and rolling circle amplification (RCA), for example. In the case of PCR based amplification, the need for rapid temperature thermocycling and purified sample restricts the use of the technology to a laboratory environment and limits the minimum cost, size and portability.

LAMP, unlike PCR, does not require rapid temperature cycling and so the power demands of the instrument are much lower. This enables a low-cost alternative to the traditional lab-based PCR thermocycler. In addition, LAMP has a short time to positivity—as fast as 5 minutes for strongly positive samples and the degree of sample purity required is much lower while still having analytical sensitivity comparable or superior to PCR. In order to detect RNA, a LAMP based system requires an enzyme or enzymes that can reverse transcribe the RNA template before LAMP amplification and detection. The RT-LAMP assay can therefore be either 1-step or 2-step, with the first step being a dedicated reverse transcriptase enzyme copying the RNA template into cDNA followed by the geometric LAMP amplification of the target, or preferably a single enzyme RT-LAMP process such as the Lava LAMP™ enzyme from Lucigen Inc., Middleton, Wis.

Upper respiratory tract infections are usually detected by taking swabs from the nasal, nasopharyngeal or throat and eluting the virus from them. The preparation of the RNA for detection by PCR requires further purification to remove contaminants that are less inhibitory to LAMP reactions. This enables a rapid and easy sample preparation for LAMP based assays—a requirement for simple point of care use. In the case of swab, directly eluting the virus into a suitable assay buffer and directly putting that sample into the molecular test system with a simple transfer step is enabling for point of care operation.

For a point of care device, speed and simplicity of use are requirements. No precise measuring during operation or requirements for environmental temperature and humidity are preferred, and the reagents should ideally not require freezing or refrigerated storage. An all in one instrument having tight temperature control, automatic fluidic staging and real time monitoring of the LAMP reaction and with software to analyze the reaction and report the results to the user is preferred. Bringing the speed and sensitivity of LAMP together with an automated system that is designed to allow for operation outside of a laboratory with simple to use operating steps and room temperature reagents, is a powerful point of care combination. A single-step enzyme RT-LAMP system reduces assay time as reverse transcription and LAMP amplification occur simultaneously and allows for detection of RNA based pathogens including the majority of respiratory viruses such as influenza A and B, coronaviruses including SARS-CoV-2, and Respiratory Syncytial Virus (RSV).

A system that was able to look for a panel of multiple potential virus pathogens from a single sample would enable definitive diagnosis of the common early upper respiratory symptoms; sore throat, cough, mild fever and running nose to distinguish serious infections such as Sars-CoV-2 or influenza from mild disease caused by rhinovirus or adenovirus, for example. The Tangen GeneSpark™ instrument was designed with all these features in mind—rapid highly accurate LAMP amplification detection with a low-cost disposable assay disk that affords a panel of up to 32 different pathogen targets from a single patient sample and portability, connectivity, and ease of use to allow for point of care results. The SARS-Cov-2 pandemic has underscored the pressing need for rapid accurate testing outside of the laboratory setting at the point of care, with the information getting immediately the patient so they can manage their exposure to others, as well delivering the result to public health databases, so that the pandemic can be tracked, traced, and controlled.

The inventions described herein meet these unsolved challenges and needs. As described in detail herein below, novel embodiments of the invention described are useful for the detection of bacteria and viruses using novel NAAT and primers. The inventions have other benefits, including significant improvements to the reaction sensitivity and specificity and allowing fewer primer designs to be developed and screened for amplification reactions.

### BRIEF SUMMARY

The inventions described and claimed herein have many attributes and embodiments including, but not limited to, those set forth or described or referenced in this Brief Summary. The inventions described and claimed herein are not limited to, or by, the features or embodiments identified in this Summary, which is included for purposes of illustration only and not restriction.

Aspects of the invention relate to apparatuses, compositions, methods, and systems for detecting or quantifying a target nucleic acid in a nucleic acid sample, and in particular for detecting target nucleic acids from microorganisms and pathogens, such as bacterial and viral, in a host, patient or subject animal.

Aspects of the invention relate to system, method and apparatus for extracting particles from biological fluids (mammalian blood, saliva, urine, nasopharyngeal fluid, bronchoalveolar lavage, and other fluids, such as from other biological matrices, etc.). Once extracted, the particles may be further processed to identify the presence (or absence) of a disease. The further processing of the particles may include, lysing a cell associated with the particle to extract the cells nucleic acid and sequencing the cell's nucleic acid to determine its identity. As referenced herein, particles may include, but are not limited to, pathogens, such as bacterial and viral microorganisms.

Aspects of the invention also include kits for detecting or quantifying a target nucleic acid in a blood sample. An exemplary kit includes (i) a blood processing unit to extract particles from mammalian blood; (ii) sample prep section for separating nucleic acid associated with the extracted particles; (iii) a solid phase disc for identifying nucleic acids having one or more amplification primer sets and one or more second primer sets; and (iv) instructions for use of the disc for a method of detecting a microorganism in a nucleic acid sample from a subject on an apparatus, instrument, or system described herein or in related applications.

Other aspects of the invention to system, method and apparatus for extracting particles from body fluids (e.g. saliva, mucus, urine, oral swabs, nasal swabs, etc.). Once extracted, the particles may be further processed to identify the presence (or absence) of a disease. The further processing of the particles may include, lysing a cell associated with the particle to extract the cells nucleic acid and sequencing the cell's nucleic acid to determine its identity. As referenced herein, particles may include, but are not limited to, pathogens, such as bacterial and viral microorganisms.

In another aspect, methods of detecting a nucleic acid of one or more microorganism in a subject are provided. The sample collection and other steps of these methods may vary depending on the type of tissue sample that is being collected and what microorganism is suspected of being present.

An exemplary embodiment of a method of detecting a nucleic acid of one or more microorganism in a subject includes, independent of order, the following steps: obtaining an upper respiratory sample from a subject; processing the upper respiratory sample in an apparatus to capture and lyse microorganisms from the sample, and obtaining a nucleic acid extract from microorganisms in the upper respiratory sample of a subject; selecting one or more target sequence from a microorganism of interest, and selecting one or more nucleic acid amplification primer set that is complementary to at least a portion of a target sequence from a microorganism of interest; incubating the target sequence with the one or more nucleic acid amplification primer set in a reaction mixture and performing an amplification reaction; and detecting one or more target sequence from a microorganism of interest.

In some embodiments, the incubation step includes a pre-amplification step that uses random primers and reagents for the nonselective amplification of nucleic acid from microorganisms in the sample to produce a pre-amplification product.

In some embodiments, an upper respiratory sample from a subject includes samples from a nasal pharyngeal swab, a nasal swab, a throat swab, saliva, a nasal aspirate, and any other method suitable to obtain sufficient sample. In some embodiments, more than one microorganism in a subject's upper respiratory sample can be detected.

In some embodiments, the microorganism detected is a virus. In some embodiments, the virus is a coronavirus. In some embodiments, the virus is a SARS-CoV-2 type virus. In some embodiments, the virus is selected from Adenovirus, Coronavirus HKU1, Coronavirus NL63, Coronavirus 229E, Coronavirus OC43, Human Metapneumovirus, Human Rhinovirus/Enterovirus, Influenza A, Influenza B, Parainfluenza Virus 1, Parainfluenza Virus 2, Parainfluenza Virus 3, Parainfluenza Virus 4Respiratory Syncytial Virus, and SARS-CoV-2 type virus.

In some embodiments, the amplified template is detected or quantified in real time. In some embodiments, between about 100 and about 1000 of amplicon products from a first stage amplification are inputted into a second stage amplification reaction. In some embodiments, the amplification is isothermal.

In some embodiments, the target sequence comprises a SARS-CoV-2 type virus nucleic acid sequence in the nucleocapsid recombinant N2 fragment domain or in the nucleocapsid recombinant N3 fragment domain. In some embodiments, the target sequence comprises a SARS-CoV-2 type virus nucleic acid sequence in the nucleocapsid recombinant N2 fragment domain the target sequence comprises a nucleocapsid recombinant N3 fragment domain.

Another exemplary embodiment of a method of detecting a nucleic acid of one or more microorganism in a subject includes, independent of order, the following steps: obtaining a blood or blood fraction sample from a subject; processing the blood sample in an apparatus to capture and lyse microorganisms from the sample, and obtaining a nucleic acid extract from microorganisms in the blood sample of a subject; selecting one or more target sequence from a microorganism of interest, and selecting one or more nucleic acid amplification primer set that is complementary to at least a portion of a target sequence from a microorganism of interest; incubating the target sequence with the one or more nucleic acid amplification primer set in a reaction mixture and performing an amplification reaction; and detecting one or more target sequence from a microorganism of interest. In some iterations, the incubation step includes a pre-amplification step that uses random primers and reagents for the nonselective amplification of nucleic acid from microorganisms in the sample to produce a pre-amplification product.

In some embodiments, more than one microorganism in a subject's blood sample can be detected. In some embodiments, the microorganism comprises one or more bacteria species.

In some embodiments, one or more bacteria species that is detected is selected from Bordetella parapertussis, Bordetella pertussis, Chlamydia pneumoniae, Mycoplasma pneumoniae, Escherichia coli, Klebsiella pneumoniae, Klebsiella oxytoca, Salmonella spp., Proteus mirabilis, Citrobacter freundii, Serratia marcescens, Enterococcus faecalis, Enterococcus faecium, Staphylococcus aureus, Staphylococcus epidermidis, Staphylococcus lugdunensis, and Streptococcus pneumoniae.

In some embodiments, one or more bacterium species is a pathogenic bacterium.

In some embodiments, the bacterium Bacillus anthracis is detected and the target nucleic acids comprise pXO1 and pXO2 nucleic acid sequences from bacterium B. anthracis.

In some embodiments, a target sequence comprises nucleic acids from genes that confer antimicrobial resistance (AMR) to bacteria. In some embodiments, one or more antimicrobial resistance gene (AMR) is detected from one or more bacterial pathogen species suspected of being present in a subject's blood sample. In some embodiments, at least 10 species of bacteria and their corresponding antibiotic resistance genes are analyzed. In some embodiments, between about 10 and about 20 species of bacteria and their corresponding antibiotic resistance genes are analyzed. In some embodiments, the amplified template is detected or quantified in real time. In some embodiments, between about 100 and about 1000 of amplicon products from a first stage amplification are inputted into a second stage amplification reaction. In some embodiments, the amplification is isothermal.

The invention also includes kits for detecting or quantifying a target nucleic acid in a nucleic acid sample, an exemplary kit includes a solid phase disc for detecting nucleic acids having one or more amplification primer sets and one or more second primer sets; and ii) instructions for use of the disk for a method of detecting a microorganism in a nucleic acid sample from a subject on an apparatus, instrument, or system described herein.

### BRIEF DESCRIPTION OF THE DRAWINGS

FIG. 1A and FIG. 1B show an embodiment used for the detection of Candida auris.

FIG. 1A illustrates the primers used for LAMP reaction and their location in the target sequence. FIG. 1B is an illustration of the Candida auris TangenDX™ assay disks. Wells in black contained positive controls, wells in grey contain the Candida auris LAMP primers, and wells in white are empty. The arrangement/assignment of the wells does not have be in consecutive order.

FIG. 2A and FIG. 2B show the distribution of the quantification cycles (Cq) (FIG. 2A) and percentage of positive wells (PPW) (FIG. 2B) for each set of LAMP primers tested. Dark grey circles indicate the results for blank runs and light grey circles results for runs containing 2,000 genomes of C. auris M5658.

FIG. 3 shows components of a TangenDx™-Candida auris assay, which is provided as a kit in some embodiments. Such a kit may include all or some of the following: a BD ESwab collection and transport system (1); a capped 20 mL syringe prefilled with 5 mL of lysis buffer (2); a 2.5″ 18-gauge blunt needle (3); a large volume concentrator (LVC) unit attached to an LVC-adaptor (4); a capped 20 mL syringe prefilled with 12 mL of wash buffer (5); a bottle with 10 mL assay buffer (6) a 4004 transfer pipette (7); a LVC cap with a lyticase enzyme bead (8); a LVC holder (9); a LVC tightening wrench (10); and a waste container (11).

FIG. 4 shows a comparison of percentage of positive wells (PPW) with and without filtration.

FIG. 5 shows results of a LoD Range Finding-Reference Method Comparison experiment.

FIG. 6 shows more results of a LoD Range Finding-Reference Method Comparison experiment.

### DETAILED DESCRIPTION

Various aspects of the invention will now be described with reference to the following section which will be understood to be provided by way of illustration only and not to constitute a limitation on the scope of the invention.

“Complementarity” refers to the ability of a nucleic acid to form hydrogen bond(s) or hybridize with another nucleic acid sequence by either traditional Watson-Crick or other non-traditional types. As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA. See e.g. Ausubel, et al., Current Protocols In Molecular Biology, John Wiley & Sons, New York, N.Y., 1993. If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are “substantially complementary” to each other when a sufficient number of corresponding positions in each molecule are occupied by nucleotides that can hybridize or anneal with each other in order to affect the desired process. A complementary sequence is a sequence capable of annealing under stringent conditions to provide a 3′-terminal serving as the origin of synthesis of complementary chain.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, Md.). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

The terms “amplify”, “amplifying”, “amplification reaction”, or a “NAAT” and their variants, refer generally to any action or process whereby at least a portion of a nucleic acid molecule (referred to as a template nucleic acid molecule) is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. In some embodiments, amplification includes a template-dependent in vitro enzyme-catalyzed reaction for the production of at least one copy of at least some portion of the nucleic acid molecule or the production of at least one copy of a nucleic acid sequence that is complementary to at least some portion of the nucleic acid molecule. Amplification optionally includes linear or exponential replication of a nucleic acid molecule. In some embodiments, such amplification is performed using isothermal conditions; in other embodiments, such amplification can include thermocycling. In some embodiments, the amplification is a multiplex amplification that includes the simultaneous amplification of a plurality of target sequences in a single amplification reaction. At least some of the target sequences can be situated, on the same nucleic acid molecule or on different target nucleic acid molecules included in the single amplification reaction. In some embodiments, “amplification” includes amplification of at least some portion of DNA- and RNA-based nucleic acids alone, or in combination. The amplification reaction can include single or double-stranded nucleic acid substrates and can further including any of the amplification processes known to one of ordinary skill in the art. In some embodiments, the amplification reaction includes polymerase chain reaction (PCR). In the present invention, the terms “synthesis” and “amplification” of nucleic acid are used. The synthesis of nucleic acid in the present invention means the elongation or extension of nucleic acid from an oligonucleotide serving as the origin of synthesis. If not only this synthesis but also the formation of other nucleic acid and the elongation or extension reaction of this formed nucleic acid occur continuously, a series of these reactions is comprehensively called amplification.

The terms “target primer” or “target-specific primer” and variations thereof refer to primers that are complementary to a binding site sequence. Target primers are generally a single stranded or double-stranded polynucleotide, typically an oligonucleotide, that includes at least one sequence that is at least partially complementary to a target nucleic acid sequence.

“Forward primer binding site” and “reverse primer binding site” refers to the regions on the template DNA and/or the amplicon to which the forward and reverse primers bind. The primers act to delimit the region of the original template polynucleotide which is exponentially amplified during amplification. In some embodiments, additional primers may bind to the region 5′ of the forward primer and/or reverse primers. Where such additional primers are used, the forward primer binding site and/or the reverse primer binding site may encompass the binding regions of these additional primers as well as the binding regions of the primers themselves. For example, in some embodiments, the method may use one or more additional primers which bind to a region that lies 5′ of the forward and/or reverse primer binding region. Such a method was disclosed, for example, in WO0028082 which discloses the use of “displacement primers” or “outer primers”.

In some embodiments, amplification can be performed using multiple target-specific primer pairs in a single amplification reaction, wherein each primer pair includes a forward target-specific primer and a reverse target-specific primer, each including at least one sequence that substantially complementary or substantially identical to a corresponding target sequence in the sample, and each primer pair having a different corresponding target sequence. In some embodiments, the target-specific primer can be substantially non-complementary at its 3′ end or its 5′ end to any other target-specific primer present in an amplification reaction. In some embodiments, the target-specific primer can include minimal cross hybridization to other target-specific primers in the amplification reaction. In some embodiments, target-specific primers include minimal cross-hybridization to non-specific sequences in the amplification reaction mixture. In some embodiments, the target-specific primers include minimal self-complementarity. In some embodiments, the target-specific primers can include one or more cleavable groups located at the 3′ end. In some embodiments, the target-specific primers can include one or more cleavable groups located near or about a central nucleotide of the target-specific primer. In some embodiments, one of more targets-specific primers includes only non-cleavable nucleotides at the 5′ end of the target-specific primer. In some embodiments, a target specific primer includes minimal nucleotide sequence overlap at the 3′end or the 5′ end of the primer as compared to one or more different target-specific primers, optionally in the same amplification reaction. In some embodiments 1, 2, 3, 4, 5, 6, 7, 8, 9, 10 or more, target-specific primers in a single reaction mixture include one or more of the above embodiments. In some embodiments, substantially all of the plurality of target-specific primers in a single reaction mixture includes one or more of the above embodiments.

The terms “identity” and “identical” and their variants, as used herein, when used in reference to two or more nucleic acid sequences, refer to similarity in sequence of the two or more sequences (e.g., nucleotide or polypeptide sequences). In the context of two or more homologous sequences, the percent identity or homology of the sequences or subsequences thereof indicates the percentage of all monomeric units (e.g., nucleotides or amino acids) that are the same (i.e., about 70% identity, preferably 75%, 80%, 85%, 90%, 95%, 97%, 98% or 99% identity). The percent identity can be over a specified region, when compared and aligned for maximum correspondence over a comparison window, or designated region as measured using a BLAST or BLAST 2.0 sequence comparison algorithms with default parameters described below, or by manual alignment and visual inspection. Sequences are said to be “substantially identical” when there is at least 85% identity at the amino acid level or at the nucleotide level. Preferably, the identity exists over a region that is at least about 25, 50, or 100 residues in length, or across the entire length of at least one compared sequence. A typical algorithm for determining percent sequence identity and sequence similarity are the BLAST and BLAST 2.0 algorithms, which are described in Altschul et al, Nuc. Acids Res. 25:3389-3402 (1977). Other methods include the algorithms of Smith & Waterman, Adv. Appl. Math. 2:482 (1981), and Needleman & Wunsch, J. Mol. Biol. 48:443 (1970), etc. Another indication that two nucleic acid sequences are substantially identical is that the two molecules or their complements hybridize to each other under stringent hybridization conditions.

The polynucleic acid produced by an amplification technology employed is generically referred to as an “amplicon” or “amplification product.” The nature of amplicon produced varies significantly depending on the NAAT being practiced. For example, NAATs such as PCR may produce amplicon which is substantially of identical size and sequence. Other NAATs produce amplicon of very varied size wherein the amplicon is composed of different numbers of repeated sequences such that the amplicon is a collection of concatamers of different length. The repeating sequence from such concatamers will reflect the sequence of the polynucleic acid which is the subject of the assay being performed. In the present specification, the simple expression “5′-side” or “3′-side” refers to that of a nucleic acid chain serving as a template, wherein the 5′ end generally includes a phosphate group and a 3′ end generally includes a free —OH group.

### Apparatus

In some embodiments, an apparatus and methods for rapid isolation, concentration, and purification of microbes/pathogens of interest from a raw biological sample such as blood is described. Samples may be processed directly from biological or clinical sample collection vessels, such as vacutainers, by coupling with the sample processing apparatus in such a manner that minimizes or eliminates user exposure and potential contamination issues. In various embodiments, the apparatus comprises a staged syringe or piston arrangement configured to withdraw a desired quantity of biological sample from a sample collection vessel. The sample is then mixed with selected processing reagents preparing the sample for isolation of microbes or pathogens contained therein. Sample processing may include liquefying or homogenizing non-pathogenic components of the biological specimen and performing various fluidic transfer operations induced by operation of the syringe or piston. The resulting sample constituents may be redirected to flow across a capture filter or membrane of appropriate size or composition to capture specific microbes/pathogens or other biological sample constituents. Additional operations may be performed including washing and drying of the filter or membrane by action of the syringe or piston. In various embodiments, sample backflow and cross-contamination within the device is avoided using one-way valves that direct sample fluids along desired paths while preventing leakage, backflow, and/or undesired sample movement.

The device may include a capture filter for retaining microbes/pathogens of interest allowing them to be readily separated from sample eluent or remaining fraction of the processed sample/waste. The capture filter may be housed in a sealable container and can further be configured to be received directly by other sample processing/analytical instruments for performing downstream operations such as lysis, elution, detection, and identification of the captured microbes/pathogens retained on the filter/membrane.

The collector may comprise various features to facilitate automated or semi-automated sample processing and include additional reagents contained in at least one reservoir integrated into the collector to preserve or further process the isolated microbes/pathogens captured or contained by the filter/membrane. In various embodiments, the collector may contain constituents capable of chemically disinfecting the isolated microbes/pathogens or render the sample non-infectious while preserving the integrity of biological constituents associated with the microbe/pathogen such as nucleic acids and/or proteins that may be desirably isolated for subsequent downstream processing and analysis. The collector and associated instrument components may desirably maintain the sample in an isolated environment avoiding sample contamination and/or user exposure to the sample contents.

In various embodiments, this present disclosure describes an apparatus that permits rapid and semi-automated isolation and extraction of microorganisms such as bacteria, virus, spores, and fungi or constituent biomolecules associated with the microorganisms, such as nucleic acids and/or proteins from a biological sample without extensive hands-on processing or lab equipment. The apparatus has the further benefit of concentrating the microbes, pathogens, or associated biomolecules/biomaterial of interest. For example, bacteria, virus, spores, or fungi present in the sample (or nucleic acids and/or proteins associated therewith) may be conveniently isolated from the original sample material and concentrated on the filter or membrane. Concentration in this manner increases the efficiency of the downstream assays and analysis improving detection sensitivity by providing lower limits of detection relative to the input sample.

The sample preparation apparatus of the present disclosure may further be adapted for use with analytical devices and instruments capable of processing and identifying the microorganisms and/or associated biomolecules present within the biological sample. In various embodiments, the sample collector and various other components of the system can be fabricated from inexpensive and disposable materials such as molded plastic that are compatible with downstream sample processing methods and economical to produce. Such components may be desirably sealed and delivered in a sterile package for single use thereby avoiding potential contamination of the sample contents or exposure of the user while handling. In various embodiments, the reagents of the sample collector provide for disinfection of the sample constituents such that may be disposed of without risk or remaining infectious or hazardous. The sample collector provides simplified workflows and does not require specialized training or procedures for handling and disposal.

In various embodiments, the automated and semi-automated processing capabilities of the system simplify sample preparation and processing protocols. A practical benefit may be realized in an overall reduction in the number of required user operations, interactions, or potential sample exposures as compared to conventional sample processing systems. This results in lower user training requirements and fewer user-induced failure points. In still other embodiments, the system advantageously provides effective isolation and/or decontamination of a sample improving overall user safety while at the same time preserving sample integrity, for example by reducing undesirable sample degradation. Further aspects of these embodiments are described in co-pending related applications.

In some embodiments, an apparatus and methods for rapid isolation, concentration, and purification of microbes/pathogens of interest from a raw biological sample such as blood is described. Samples may be processed directly from biological or clinical sample collection vessels, such as vacutainers, by coupling with the sample processing apparatus in such a manner that minimizes or eliminates user exposure and potential contamination issues. In various embodiments, the apparatus comprises a staged syringe or piston arrangement configured to withdraw a desired quantity of biological sample from a sample collection vessel. The sample is then mixed with selected processing reagents preparing the sample for isolation of microbes or pathogens contained therein. Sample processing may include liquefying or homogenizing non-pathogenic components of the biological specimen and performing various fluidic transfer operations induced by operation of the syringe or piston. The resulting sample constituents may be redirected to flow across a capture filter or membrane of appropriate size or composition to capture specific microbes/pathogens or other biological sample constituents. Additional operations may be performed including washing and drying of the filter or membrane by action of the syringe or piston. In various embodiments, sample backflow and cross-contamination within the device is avoided using one-way valves that direct sample fluids along desired paths while preventing leakage, backflow, and/or undesired sample movement.

The device may include a capture filter for retaining microbes/pathogens of interest allowing them to be readily separated from sample eluent or remaining fraction of the processed sample/waste. The capture filter may be housed in a sealable container and can further be configured to be received directly by other sample processing/analytical instruments for performing downstream operations such as lysis, elution, detection, and identification of the captured microbes/pathogens retained on the filter/membrane.

The collector may comprise various features to facilitate automated or semi-automated sample processing and include additional reagents contained in at least one reservoir integrated into the collector to preserve or further process the isolated microbes/pathogens captured or contained by the filter/membrane. In various embodiments, the collector may contain constituents capable of chemically disinfecting the isolated microbes/pathogens or render the sample non-infectious while preserving the integrity of biological constituents associated with the microbe/pathogen such as nucleic acids and/or proteins that may be desirably isolated for subsequent downstream processing and analysis. The collector and associated instrument components may desirably maintain the sample in an isolated environment avoiding sample contamination and/or user exposure to the sample contents.

In various embodiments, this present disclosure describes an apparatus that permits rapid and semi-automated isolation and extraction of microorganisms such as bacteria, virus, spores, and fungi or constituent biomolecules associated with the microorganisms, such as nucleic acids and/or proteins from a biological sample without extensive hands-on processing or lab equipment. The apparatus has the further benefit of concentrating the microbes, pathogens, or associated biomolecules/biomaterial of interest. For example, bacteria, virus, spores, or fungi present in the sample (or nucleic acids and/or proteins associated therewith) may be conveniently isolated from the original sample material and concentrated on the filter or membrane. Concentration in this manner increases the efficiency of the downstream assays and analysis improving detection sensitivity by providing lower limits of detection relative to the input sample.

The sample preparation apparatus of the present disclosure may further be adapted for use with analytical devices and instruments capable of processing and identifying the microorganisms and/or associated biomolecules present within the biological sample. In various embodiments, the sample collector and various other components of the system can be fabricated from inexpensive and disposable materials such as molded plastic that are compatible with downstream sample processing methods and economical to produce. Such components may be desirably sealed and delivered in a sterile package for single use thereby avoiding potential contamination of the sample contents or exposure of the user while handling. In various embodiments, the reagents of the sample collector provide for disinfection of the sample constituents such that may be disposed of without risk or remaining infectious or hazardous. The sample collector provides simplified workflows and does not require specialized training or procedures for handling and disposal.

In various embodiments, the automated and semi-automated processing capabilities of the system simplify sample preparation and processing protocols. A practical benefit may be realized in an overall reduction in the number of required user operations, interactions, or potential sample exposures as compared to conventional sample processing systems. This results in lower user training requirements and fewer user-induced failure points. In still other embodiments, the system advantageously provides effective isolation and/or decontamination of a sample improving overall user safety while at the same time preserving sample integrity, for example by reducing undesirable sample degradation.

### Methods

In some embodiments, more than one amplification is performed and the separate amplifications are referenced herein as stages or stages of amplification. Unless explicitly expressed otherwise, any of the amplification techniques or NAAT's described herein can be used in combination in some embodiments of the methods of increasing the performance and specificity of amplification reactions described herein. Thus, an isothermal type amplification reaction such as LAMP can be combined with a non-isothermal amplification such as PCR, or as another example, another isothermal amplification such as a Helicase Dependent Amplification (HAD) reaction. In these embodiments, the amplification that is performed first sequentially is the first-stage amplification reaction, the amplification that is performed second sequentially is termed the second-stage amplification reaction, the amplification that is performed third sequentially is termed the third-stage amplification reaction, and so on. The inventors envision that any combination of NAAT's can be used in two-stage, three-stage, four-stage, or other multi-stage amplification embodiments of the invention described and provided herein.

A number of isothermal amplification techniques (iNAATs) can be utilized in embodiments of the invention. Many of these approaches are mentioned above, and some in particular will be described in greater detail. Isothermal amplification techniques typically utilize DNA polymerases with strand-displacement activity, thus eliminating the high temperature melt cycle that is required for PCR. This allows isothermal techniques to be faster and more energy efficient than PCR, and also allows for simpler and lower cost instrumentation since rapid temperature cycling is not required. For example, some methods of the instant invention are directed toward the improvement of conventional iNAAT's such as Strand Displacement Amplification (SDA; G. T. Walker, et at. 1992. Proc. Natl. Acad. Sci. USA 89, 392-396; G. T. Walker, et al. 1992. Nuc. Acids. Res. 20, 1691-1696; U.S. Pat. No. 5,648,211 and EP 0 497 272, all disclosures being incorporated herein by reference); self-sustained sequence replication (35R; J. C. Guatelli, et al. 1990. Proc. Natl. Acad. Sci. USA 87, 1874-1878, which is incorporated herein by reference); and Q.beta. replicase system (P. M. Lizardi, et al. 1988. BioTechnology 6, 1197-1202, which is incorporated herein by reference) are isothermal reactions (See also, Nucleic Acid Isothermal Amplification Technologies—A Review. Nucleosides, Nucleotides and Nucleic Acids, 2008. v27(3):224-243, which is incorporated herein by reference).

Some isothermal amplification techniques are dependent on transcription as part of the amplification process, for example Nucleic Acid Sequence Based Amplification (NASBA; U.S. Pat. No. 5,409,818) and Transcription Mediated Amplification (TMA; U.S. Pat. No. 5,399,491) while others are dependent on the action of a Helicase or Recombinase for example Helicase Dependent Amplification (HDA; WO2004027025) and Recombinase Polymerase Amplification (RPA; WO03072805) respectively, others still are dependent on the strand displacement activity of certain DNA polymerases, for example Strand Displacement Amplification (SDA; U.S. Pat. No. 5,455,166), Loop-mediated Isothermal Amplification (LAMP; WO0028082, WO0134790, WO0224902), Chimera Displacement Reaction (CDR; WO9794126), Rolling Circle Amplification (RCA; Lizardi, P. M. et al. Nature Genetics, (1998) 19.225-231), Isothermal Chimeric Amplification of Nucleic Acids (ICAN; WO0216639), SMart Amplification Process (SMAP; WO2005063977), Linear Isothermal Multimerization Amplification (LIMA; Isothermal amplification and multimerization of DNA by Bst DNA polymerase, Hafner G. J., Yang I. C., Wolter L. C., Stafford M. R., Giffard P. M, BioTechniques, 2001, vol. 30, no 4, pp. 852-867) also methods as described in U.S. Pat. No. 6,743,605 (herein referred to as ‘Template Re-priming Amplification’ or TRA) and WO9601327 (herein referred to as ‘ Self Extending Amplification’ or SEA).

The methods as described herein can be practiced with any NAAT, including non-isothermal technologies. For example, known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; U.S. Pat. Nos. 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.).

For instance, polymerase chain reaction (PCR) technology can be used to amplify the sequences of polynucleotides of the present invention and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

A common characteristic of the NAATs described herein is that they provide for both copying of a polynucleic acid via the action of a primer or set of primers and for re-copying of said copy by a reverse primer or set of primers. This enables the generation of copies of the original polynucleic acid at an exponential rate. With reference to NAATs in general it is helpful to differentiate between the physical piece of nucleic acid being detected by the method, from the first copy made of this original nucleic acid, from the first copy of the copy made from this original nucleic acid, from further copies of this copy of a copy. A nucleic acid whose origin is from the sample being analyzed itself will be referred to as the “target nucleic acid template.” With reference to the two-stage embodiments described herein, generally, but not always, the first-stage primer-dependent amplification reaction is relatively slow as compared to the second-stage reaction.

As would be understood by the skilled artisan, a primer-generated amplicon gives rise to further generations of amplicons through repeated amplification reactions of the target nucleic acid template as well as priming of the amplicons themselves. It is possible for amplicons to be comprised of combinations with the target template.

The amplicon may be of very variable length as the target template can be copied from the first priming site beyond the region of nucleic acid delineated by the primers employed in a particular NAAT. In general, a key feature of a NAAT in an embodiment herein, whether it is one-step, two-step, or multistep NAAT reaction, will be to provide a method by which the amplicon can be made available to another primer employed by the methodology so as to generate (over repeated amplification reactions) amplicons that will be of a discrete length delineated by the primers used. A key feature of the NAAT is to provide a method by which the amplicons are available for further priming by a reverse primer in order to generate further copies. For some NAATs, the later generation amplicons may be substantially different from the first-generation amplicon, in particular, the formed amplicon may be a concatamer of the first-generation amplicon.

An exemplary target template used in the present invention includes any polynucleic acid that comprises suitable primer binding regions that allow for amplification of a polynucleic acid of interest. The skilled person will understand that the forward and reverse primer binding sites need to be positioned in such a manner on the target template that the forward primer binding region and the reverse primer binding region are positioned 5′ of the sequence which is to be amplified on the sense and antisense strand, respectively. The target template may be single or double stranded. Where the target template is a single stranded polynucleic acid, the skilled person will understand that the target template will initially comprise only one primer binding region. However, the binding of the first primer will result in synthesis of a complementary strand which will then contain the second primer binding region. The target template may be derived from an RNA molecule, in which case the RNA needs to be transcribed into DNA before practicing the method of the invention. Suitable reagents for transcribing the RNA are well known in the art and include, but are not limited to, reverse transcriptase.

The terms “nucleic acid,” “polynucleotides,” and “oligonucleotides” refers to biopolymers of nucleotides and, unless the context indicates otherwise, includes modified and unmodified nucleotides, and both DNA and RNA, and modified nucleic acid backbones. For example, in certain embodiments, the nucleic acid is a peptide nucleic acid (PNA) or a locked nucleic acid (LNA). Typically, the methods as described herein are performed using DNA as the nucleic acid template for amplification. However, nucleic acid whose nucleotide is replaced by an artificial derivative or modified nucleic acid from natural DNA or RNA is also included in the nucleic acid of the present invention insofar as it functions as a template for synthesis of complementary chain. The nucleic acid of the present invention is generally contained in a biological sample. The biological sample includes animal, plant or microbial tissues, cells, cultures, and excretions, or extracts therefrom. In certain aspects, the biological sample includes intracellular parasitic genomic DNA or RNA such as virus or mycoplasma. The nucleic acid may be derived from nucleic acid contained in said biological sample. For example, genomic DNA, or cDNA synthesized from mRNA, or nucleic acid amplified on the basis of nucleic acid derived from the biological sample, are preferably used in the described methods. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes thymidine, and “U’ denotes deoxyuridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

A template nucleic acid in exemplary embodiments is a nucleic acid serving as a template for synthesizing a complementary chain in a nucleic acid amplification technique. A complementary chain having a nucleotide sequence complementary to the template has a meaning as a chain corresponding to the template, but the relationship between the two is merely relative. That is, according to the methods described herein a chain synthesized as the complementary chain can function again as a template. That is, the complementary chain can become a template. In certain embodiments, the template is derived from a biological sample, e.g., plant, animal, virus, micro-organism, bacteria, fungus, etc. In certain embodiments, the animal is a mammal, e.g., a human patient.

A template nucleic acid typically comprises one or more target nucleic acid. A target nucleic acid in exemplary embodiments may comprise any single or double-stranded nucleic acid sequence that can be amplified or synthesized according to the disclosure, including any nucleic acid sequence suspected or expected to be present in a sample. In some embodiments, the target sequence is present in double-stranded form and includes at least a portion of the particular nucleotide sequence to be amplified or synthesized, or its complement, prior to the addition of target-specific primers or appended adapters. Target sequences can include the nucleic acids to which primers useful in the amplification or synthesis reaction can hybridize prior to extension by a polymerase. In some embodiments, the term refers to a nucleic acid sequence whose sequence identity, ordering or location of nucleotides is determined by one or more of the methods of the disclosure.

A primer pair for a target nucleic acid typically has at least a region that is complementary to a target nucleic acid template in the sample. NAAT primers used in the compositions, methods, and other inventions described herein typically at least 75% complementary or at least 85% complementary, more typically at least 90% complementary, more typically at least 95% complementary, more typically at least 98% or at least 99% complementary, or identical, to at least a portion of a nucleic acid molecule that includes a target sequence. In such instances, the target primer or target-specific primer and target sequence are described as “corresponding” to each other. In some embodiments, the target-specific primer is capable of hybridizing to at least a portion of its corresponding target sequence (or to a complement of the target sequence); such hybridization can optionally be performed under standard hybridization conditions or under stringent hybridization conditions. In some embodiments, the target-specific primer is not capable of hybridizing to the target sequence, or to its complement, but is capable of hybridizing to a portion of a nucleic acid strand including the target sequence, or to its complement.

In some embodiments, the target-specific primer includes at least one sequence that is at least 75% complementary, typically at least 85% complementary, more typically at least 90% complementary, more typically at least 95% complementary, more typically at least 98% complementary, or more typically at least 99% complementary, to at least a portion of the target sequence itself; in other embodiments, the target-specific primer includes at least one sequence that is at least 75% complementary, typically at least 85% complementary, more typically at least 90% complementary, more typically at least 95% complementary, more typically at least 98% complementary, or more typically at least 99% complementary, to at least a portion of the nucleic acid molecule other than the target sequence. In some embodiments, the target-specific primer is substantially non-complementary to other target sequences present in the sample; optionally, the target-specific primer is substantially non-complementary to other nucleic acid molecules present in the sample. In some embodiments, nucleic acid molecules present in the sample that do not include or correspond to a target sequence (or to a complement of the target sequence) are referred to as “non-specific” sequences or “non-specific nucleic acids”. In some embodiments, the target-specific primer is designed to include a nucleotide sequence that is substantially complementary to at least a portion of its corresponding target sequence. In some embodiments, a target-specific primer is at least 95% complementary, or at least 99% complementary, or identical, across its entire length to at least a portion of a nucleic acid molecule that includes its corresponding target sequence. In some embodiments, a target-specific primer can be at least 90%, at least 95% complementary, at least 98% complementary or at least 99% complementary, or identical, across its entire length to at least a portion of its corresponding target sequence. In some embodiments, a forward target-specific primer and a reverse target-specific primer define a target-specific primer pair that can be used to amplify the target sequence via template-dependent primer extension.

In other embodiments, the primer comprises one or more mismatched nucleotides (i.e., bases that are not complementary to the binding site). In still other embodiments, the primer can comprise a segment that does not anneal to the polynucleic acid or that is complementary to the inverse strand of the polynucleic acid to which the primer anneals. In certain embodiments, a primer is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more nucleotides in length. In a preferred embodiment, the primer comprises from 2 to 100 nucleotides. In some embodiments, primer lengths are in the range of about 10 to about 60 nucleotides, about 12 to about 50 nucleotides, about 15 to about 50 nucleotides, about 18 to 50 nucleotides in length, about 6 to 50 nucleotides in length, about 10 to about 40 nucleotides in length, about 15 to about 40 nucleotides in length, about 18 to 40 nucleotides in length, or a different length. Typically, a primer is capable of hybridizing to a corresponding target sequence and undergoing primer extension when exposed to amplification conditions in the presence of dNTPS and a polymerase. In some instances, the particular nucleotide sequence or a portion of the primer is known at the outset of the amplification reaction or can be determined by one or more of the methods disclosed herein. In some embodiments, the primer includes one or more cleavable groups at one or more locations within the primer.

Primers and oligonucleotides used in embodiments herein comprise nucleotides. A nucleotide comprises any compound, including without limitation any naturally occurring nucleotide or analog thereof, which can bind selectively to, or can be polymerized by, a polymerase. Typically, but not necessarily, selective binding of the nucleotide to the polymerase is followed by polymerization of the nucleotide into a nucleic acid strand by the polymerase; occasionally however the nucleotide may dissociate from the polymerase without becoming incorporated into the nucleic acid strand, an event referred to herein as a “non-productive” event. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some or all of such moieties.

In other embodiments, the nucleotide can optionally include a chain of phosphorus atoms comprising three, four, five, six, seven, eight, nine, ten or more phosphorus atoms. In some embodiments, the phosphorus chain can be attached to any carbon of a sugar ring, such as the 5′ carbon. The phosphorus chain can be linked to the sugar with an intervening O or S. In one embodiment, one or more phosphorus atoms in the chain can be part of a phosphate group having P and O. In another embodiment, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In one embodiment, the phosphorus atoms in the chain can have side groups having O, BH3, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in Xu, U.S. Pat. No. 7,405,281. In some embodiments, the nucleotide comprises a label and referred to herein as a “labeled nucleotide”; the label of the labeled nucleotide is referred to herein as a “nucleotide label”. In some embodiments, the label can be in the form of a fluorescent moiety (e.g. dye), luminescent moiety, or the like attached to the terminal phosphate group, i.e., the phosphate group most distal from the sugar. Some examples of nucleotides that can be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group can include sulfur substitutions for the various oxygens, e.g. α-thio-nucleotide 5′-triphosphates. For a review of nucleic acid chemistry, see: Shabarova, Z. and Bogdanov, A. Advanced Organic Chemistry of Nucleic Acids, VCH, New York, 1994.

A number of nucleic acid polymerases can be used in the NAATs utilized in certain embodiments provided herein, including any enzyme that can catalyze the polymerization of nucleotides (including analogs thereof) into a nucleic acid strand. Such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also includes fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer-based polymerase that optionally can be reactivated.

### Microorganism Detection

In some embodiments a microorganism that is detected is a virus, and in certain embodiments the virus that is detected is selected from Adenovirus, Coronavirus HKU1, Coronavirus NL63, Coronavirus 229E, Coronavirus OC43, Human Metapneumovirus, Human Rhinovirus/Enterovirus, Influenza A, Influenza B, Parainfluenza Virus 1, Parainfluenza Virus 2, Parainfluenza Virus 3, Parainfluenza Virus 4Respiratory Syncytial Virus, and SARS-CoV-2 type virus.

Other embodiments described herein may be used for the detection of a virus, bacteria, or other microorganism, including one or more of the following viruses: Adeno-Associated Virus Parvovirus ‘AAV’, Adenovirus, Arena virus (Lassa virus), Astrovirus, Bacille Calmette-Guerin ‘BDG’, BK virus (including associated with kidney transplant patients), Papovavirus, Bunyavirus, Burkett's Lymphoma (Herpes), Calicivirus, California, encephalitis (Bunyavirus), Colorado tick fever (Reovirus), Corona virus, Coronavirus, Coxsackie, Coxsackie virus A, B (Enterovirus), Crimea-Congo hemorrhagic fever (Bunyavirus), Cytomegalovirus, Cytomegaly, Dengue (Flavivirus), Diptheria (bacteria), Ebola, Ebola/Marburg hemorrhagic fever (Filoviruses), Epstein-Barr Virus ‘EBV’, Echovirus, Enterovirus, Eastern equine encephalitis ‘EEE’, Togaviruses, Encephalitis, Enterovirus, Flavi virus, Hantavirus, Bunyavirus, Hepatitis A., (Enterovirus), Hepatitis B virus (Hepadnavirus), Hepatitis C (Flavivirus), Hepatitis E (Calicivirus), Herpes, Herpes Varicella-Zoster virus, HIV Human Immunodeficiency Virus (Retrovirus), HIV-AIDS (Retrovirus), Human Papilloma Virus ‘HPV’, Cervical cancer (Papovavirus), HSV 1 Herpes Simplex I, HSV 2 Herpes Simplex II, HTLV—T-cell leukemia (Retrovirus), Influenza (Orthomyxovirus), Japanese encephalitis (Flavivirus), Kaposi's Sarcoma associated herpes virus KSHV (Herpes HHV 8), Kyusaki, Lassa Virus, Lymphocytic Choriomeningitis Virus LCV (Arenavirus), Measles (Rubella), Measles Micro (Paramyxovirus), Monkey Bites (Herpes strain HHV 7), Mononucleosis (Herpes), Morbilli, Mumps (Paramyxovirus), Norovirus, Norwalk virus (Calicivirus), Orthomyxoviruses (Influenza virus A, B, C), Papillomavirus (warts), Papova (M.S.), Papovavirus (JC—progressive multifocal leukoencephalopathy in HIV) (Papovavirus), Parainfluenza Nonsegmented (Paramyxovirus), Paramyxovirus, ParvoParvovirus (B19 virusaplastic crises in sickle cell disease), Pertussus (bacteria), Polio (Enterovirus), Poxvirus (Smallpox), Prions, Rabies (Rhabdovirus), Reovirus, Retrovirus, Rhabdovirus (Rabies), Rhinovirus, Roseola (Herpes HHV 6), Rotavirus, Respiratory Syncytial Virus (Paramyxovirus), Rubella (Togaviruses), Bunyavirus, Flavivirus, Poxvirus, Variola, Venezuelan Equine Encephalitis ‘VEE’ (Togaviruses), Wart virus (Papillomavirus), Western Equine Encephalitis “WEE’ (Togaviruses), West Nile Virus (Flavivirus), and Yellow fever (Flavivirus).

In some embodiments a microorganism that is detected is a bacteria, such as a pathogenic bacteria, and in certain embodiments the bacteria that is detected is selected from one or more of the following bacteria species: Bacillus anthraci, Bordetella parapertussis, Bordetella pertussis, Chlamydia pneumoniae, Mycoplasma pneumoniae, Escherichia Coli, Klebsiella pneumoniae, Klebsiella oxytoca, Salmonella, Proteus mirabilis, Citrobacter freundii, Serratia marcescens, Enterococcus faecalis, Enterococcus faecium, Staphylococcus aureus, Staphylococcus epidermidis, Staphylococcus lugdunensis, and Streptococcus pneumoniae.

TABLE 1
exemplary coronavirus primers

| | |
|---|---|
| CovN2_F3_72C | CTGAGGGAGCCTTGAATACACCAA (SEQ ID NO: 1) |
| CovN2_B3_72C | CGCCATTGCCAGCCATTCTAGC (SEQ ID NO: 2) |
| CovN2_FIP_72C | TCCCTTCTGCGTAGAAGCCTTTTGGC-CCCGCAATCCTGCTAACAATGCT (SEQ ID NO: 3) |
| CovN2_BIP_72C | CAGAGGCGGCAGTCAAGCCTCTTC-CCCCTACTGCTGCCTGGAGTT (SEQ ID NO: 4) |
| CovN2_LF_72C | GTTGTTCCTTGAGGAAGTTGTAGCACGA (SEQ ID NO: 5) |
| CovN2_LB_72C | CGTTCCTCATCACGTAGTCGCAACAG (SEQ ID NO: 6) |
| CovN3_F3_72C | ATGGAGAACGCAGTGGGGC (SEQ ID NO: 7) |
| CovN3_B3_72C | TCATTTTACCGTCACCACCACGAA (SEQ ID NO: 8) |
| CovN3_FIP_72C | GCCATGTTGAGTGAGAGCGGTGAACC-GCGATCAAAACAACGTCGGCC (SEQ ID NO: 9) |
| CovN3_BIP_72C | AATTCCCTCGAGGACAAGGCGTTCCA-TGGTAGCTCTTCGGTAGTAGCCAA (SEQ ID NO: 10) |
| CovN3_LF_72C | AGACGCAGTATTATTGGGTAAACCTTGG (SEQ ID NO: 11) |
| CovN3_LB_72C | ATTAACACCAATAGCAGTCCAGATGACCA (SEQ ID NO: 12) |

TABLE 2
exemplary Candida auris primer sequences

| | |
|---|---|
| F3_Caur_3 | CGGCGAGTTGTAGTCTGGA (SEQ ID NO: 13) |
| B3_Caur_3 | TCCATCACTGTACTTGTTCGCT (SEQ ID NO: 14) |
| FIP_Caur_3 | GGGCCACAGGAAGCACTAGCACAGCAGGCAAGTCCTTTGG (SEQ ID NO: 15) |
| BIP_Caur_3 | CCGACGAGTCGAGTTGTTTGGGCGGTCTCTCGCCAATATTTAGC (SEQ ID NO: 16) |
| LoopF_Caur_3 | AAAGCAGGTACGGGGCTG (SEQ ID NO: 17) |
| LoopB_Caur_3 | GCAGCTCTAAGTGGGTGGTA (SEQ ID NO: 18) |

The following Examples are included for illustration and not limitation.

### Example I

This example describes a particular embodiment of the invention directed to a Point-of-care device for the detection of Candida auris.

### LAMP Primer Design.

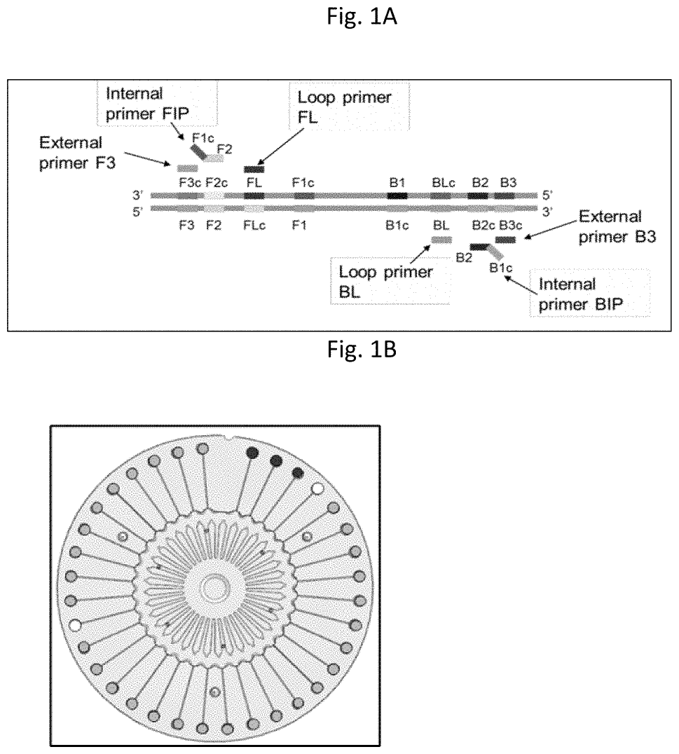

We have based our primer design in the region of the genome that codes for the ribosomal RNAs. This region is present in multiple copies in Candida species, which will help increase the limit of detection (LoD) of the assay. Each LAMP primer set consists of 3 pairs of primers, that is 6 primers in total: external primers F3 and B3, internal primers FIP and BIP and loop primers FL and BL (FIG. 1A). The criteria for primer design were: (1) to minimize the sequence mismatches with Candida auris strains from clades I, II, III, and IV; (2) maximize the sequence mismatches with other species of the genus Candida and other phylogenetically close yeast species; (3) meet the melting temperature requirements for isothermal amplification, (4) minimize the formation of secondary structures such as primer-dimers and hairpins; and (5) not cross-react with other viral, prokaryotic or eukaryotic sequences available in the public databases.

We first downloaded the whole genome sequence of all Candida auris species available in the NCBI database. We also retrieved the whole genome sequences of close Candida species including C. haemulonii, C. pseudohaemulonis and C. duobushaemulonii for the complete list of strains and accession numbers. We then used Basic Rapid Ribosomal RNA Predictor (Barrnap) to retrieve the entire ribosomal region DNA sequence for each of the sequences. We used the ribosomal region sequence of Candida auris strain B11245 (clade I) as the reference genome for design purposes. This sequence was loaded into the LAMP Designer 1.16 software (Premier Biosoft), and >150 sets of primers were designed considering the temperature and secondary structure described above (criteria 3 and 4). Each primer set was individually assessed for inclusivity with all C. auris strains (criterion 1) and specificity against C. haemulonii, C. pseudohaemulonis and C. duobushaemulonii strains (criterion 2). Primer sets fulfilling all criteria were then checked for cross-reactivity using BLAST. After this process, we obtained 6 sets of primers that met all the design criteria (Table 1).

TABLE 1
List of LAMP primer sets obtained and their location within the ribosomal
region. Candida auris strain B11245 is used as the reference for position.

| | Position (based | | |
|---|---|---|---|
| Region # | on B11245) | Gene | Sets of primers obtained |
| 1 | 631-705 | 18S | — |
| 2 | 1,73-1,845 | ITS1 | — |
| 3 | 1,999-2,089 | ITS2 | — |
| 4 | 2,234-2,236 | 28S | Designs #1, 2, and 3 |
| 5 | 2,508-2,638 | 28S | Designs #4, 5, and 6 |
| 6 | 3,564-3,656 | 28S | — |
| 7 | 3,984-4,090 | 28S | — |

We screened all six sets of LAMP primers by spotting them on TangenDx™ assay disks. Each assay disk contained 3 wells with amplification controls to assess the overall performance of the assay, 30 wells with the LAMP primer mixture (wells 5 to 24, and 26-35), and 2 empty wells for instrument calibration (wells 4 and 24). FIG. 1B shows the Candida auris TangenDX™ assay disks. Wells in black contained positive controls, wells in grey contain the Candida auris LAMP primers, and wells in white are empty.

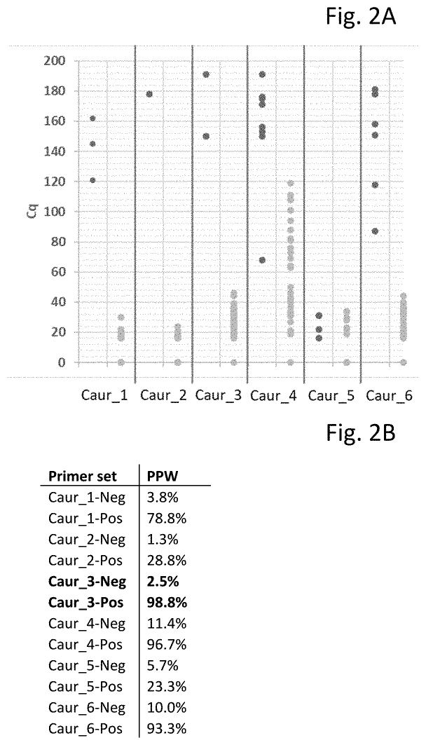

For each primer set, we run a total of 4 assay disks in the GeneSpark™ instrument with standard running conditions. The two first runs contained no DNA target (blank) and the second two runs contained 2,000 genome copies of Candida auris strain M5658 (clade I). After each run, we calculated the quantification cycle (Cq) for each well and estimated the proportion of positive wells (PPW) for each running condition (blank and 2,000 genomes). Primer set Caur_3 had the highest PPW in positive runs (98.8%) while still showing a very low PPW in blank runs (2.5%) (FIG. 2). Furthermore, the distribution of positive Cq was very narrow with all wells showing a Cq smaller than 50, while the fastest Cq for blank runs occurred at Cq=150 (FIG. 2A). All other primer sets had significantly lower PPW for positive wells, higher PPW for blank wells or overlapping Cq between positive and negative wells. Therefore, primer set Caur_3 was selected for further testing. The distribution of the quantification cycles (Cq) and percentage of positive wells (PPW) for each set of tested LAMP primers was determined. Where dark grey circles indicate the results for blank runs and light grey circles results for runs containing 2,000 genomes of C. auris M5658 (FIG. 2A).

To further characterize the performance of the Caur_3 LAMP primer set in our system, we ran additional assay disks with blank (70 wells total) and positive controls (80 wells total). All positive assay disks resulted in fast signal in the vast majority of the wells (98.8%), while no blank disks showed any signal. This result confirmed that primer set Caur_3 was specifically detecting signal from C. auris while showing no noise or background amplification (Table 2).

TABLE 2
Characterization of Caur_3 LAMP primer set using
DNA from C. auris strain M5658 and blank controls.

| | | Internal | | | |
|---|---|---|---|---|---|
| | # wells | Control | | | |
| | with | (Avg | | Avg | |
| Condition | target | Cq ± SD) | Target | Cq ± SD | PPW |
| C. auris | 80 | 50 ± 7.2 | Caur_3 | 29 ± 5.8 | 98.80% |
| M5658 | | | Detection Rate | 100% (8/8 runs) | |
| (clade I) | | | | | |
| Blank | 70 | 50 ± 4.1 | Caur_3 | NaN ± NaN | 0% |

| | |
|---|---|
| Detection Rate | 0% (0/7 runs) |

### Inclusivity Testing.

We then ran an inclusivity test with different strains of C. auris. Although Caur_3 LAMP primers were 100% identical to all C. auris strains included in the in silico analysis (see section 1 above), we confirmed this experimentally. To this end, we run Caur_3 assay disks containing 2,000 genome copies of DNA from C. auris clades II, III, and IV (strains M5655, M9897, and M8106, respectively). We observed no difference between the performance of our assay with the four C. auris clades tested (Table 3). All disks were positive, and the PPW was higher than 95% in all cases. This experimental validation demonstrated that our assay detects signal from the four C. auris clades similarly.

TABLE 3
Inclusivity analysis of Caur_3 LAMP primer set using
DNA from the four clades of C. auris.

| | | # | Internal | | | |
|---|---|---|---|---|---|---|
| | | wells | Control | | Avg | |
| | # | with | (Avg Cq ± | | Cq ± | |
| Condition | Disks | target | std dev) | Target | std dev | PPW |
| C. auris | 2 | 60 | 46 ± 11.7 | Caur_3 | 30 ± 6.8 | 95% |
| M5655 | | | | Detection | 100% (2/2 runs) | |
| (clade II) | | | | Rate | | |
| C. auris | 2 | 60 | 35 ± 5.8 | Caur_3 | 32 ± 5.4 | 100% |
| M9897 | | | | Detection | 100% (2/2 runs) | |
| (clade III) | | | | Rate | | |
| C. auris | 2 | 60 | 37 ± 3.8 | Caur_3 | 28 ± 75.2 | 100% |

| | | |
|---|---|---|
| M8106 | Detection | 100% (2/2 runs) |
| (clade IV) | Rate | |

### Specificity Testing.

To determine the specificity of the Caur_3 primer set, we run our assay with DNA extracts from species phylogenetically close to C. auris, namely C. haemulonii M5659, C. lusitaniae M240, C. duobushaemulonii M3051, C. tropicalis M57, C. albicans M48 and C. parapsilosis M40. All DNA extracts were spiked at a concentration of 2000 genomes per disk with two disks per species. All disks showed no signal for all the species tested, and no wells were positive (Table 4). This indicated that no cross-reaction occurred with close-related Candida spp. using Caur_3 primer set.

TABLE 4
Specificity testing with Caur_3 LAMP primer set using
DNA from six phylogenetically close Candida species.

| | | | Internal | | | |
|---|---|---|---|---|---|---|
| | | # wells | Control | | | |
| | # | with | (Avg Cq ± | | Avg Cq ± | |
| Condition | Disks | target | std dev) | Target | std dev | PPW |
| C. haemulonii M5659 | 2 | 60 | 46 ± 6.5 | Caur_3 | NaN ± NaN | 0% |
| | | | | Detection Rate | 0% (0/2 runs) | |
| C. lusitaniae M240 | 2 | 60 | 51 ± 5.6 | Caur_3 | NaN ± NaN | 0% |
| | | | | Detection Rate | 0% (0/2 runs) | |
| C. duobushaemulonii M3051 | 2 | 60 | 54 ± 2.8 | Caur_3 | NaN ± NaN | 0% |
| | | | | Detection Rate | 0% (0/2 runs) | |
| C. tropicalis M57 | 2 | 60 | 54 ± 2.5 | Caur_3 | NaN ± NaN | 0% |
| | | | | Detection Rate | 0% (0/2 runs) | |
| C. albicans M48 | 2 | 60 | 50 ± 5.5 | Caur_3 | NaN ± NaN | 0% |
| | | | | Detection Rate | 0% (0/2 runs) | |
| C. parapsilosis M40 | 2 | 60 | 52 ± 17.5 | Caur_3 | NaN ± NaN | 0% |

| | |
|---|---|
| Detection Rate | 0% (0/2 runs) |

Next, we tested additional DNA extracts from other Candida species and close related yeasts at the same concentrations as above. In this case, however, DNAs were pooled in groups of three for a final concentration of 6,000 genomes/disk (2,000 genomes/species). As shown in table 5, we observed no signal for any of the species tested confirming the high specificity of Caur_3 prime set.

TABLE 5
Specificity testing with Caur_3 LAMP primer set using DNA from 18 Candida
species and other yeasts.

| | | # | Internal | | | |
|---|---|---|---|---|---|---|
| | | wells | Control | | | |
| | # | with | (Avg Cq ± | | Avg Cq ± | |
| Condition | Disks | target | std dev) | Target | std dev | PPW |
| Group A (C. krusei, C. | 2 | 60 | 46 ± 2.1 | Caur_3 | NaN ± NaN | 0% |
| glabrata, C. famata) | | | | Detection | 0% (0/2 runs) | |
| Group B (C. orthopsilosis, C. | 2 | 60 | 49 ± 5.4 | Caur_3 | NaN ± NaN | 0% |
| colliculosa, C. dubliniensis) | | | | Detection | 0% (0/2 runs) | |
| Group C (C. fabianii, Cr. | 2 | 60 | 46 ± 2.3 | Caur_3 | NaN ± NaN | 0% |
| gattii, P. norvegensis) | | | | Detection | 0% (0/2 runs) | |
| Group D (Cr. albidus, D. | 2 | 60 | 52 ± 1.5 | Caur_3 | NaN ± NaN | 0% |
| hansenii, Cr. neoformans) | | | | Detection | 0% (0/2 runs) | |
| Group E (C. blankii, C. ciferrii | 2 | 60 | 47 ± 6.7 | Caur_3 | NaN ± NaN | 0% |
| Cr. diffluens) | | | | Detection | 0% (0/2 runs) | |
| Group F (S. cerevisiae, M | 2 | 60 | 48 ± 5.5 | Caur_3 | NaN ± NaN | 0% |

| | | |
|---|---|---|
| pachydermata and M. fufur) | Detection | 0% (0/2 runs) |

Sensitivity and Analytical Limit of Detection (LoD). Finally, we estimated the sensitivity and LoD of the Caur_3 primer set. We ran experiments at low input concentrations of C. auris M5658 (5 or 10 copies per disk). A shown in Table 6, an input of 10 genomes per disk resulted in positive disks with PPW of approximately 50%. This indicates that the sensitivity of the Caur_3 primer set is approximately 10 genomes per disk (sensitivity defined as the number of genomes required to produce 15 positives per disk with 30 wells dedicated to the primer set). An input of 5 genomes per disk resulted in a PPW of approximately 25%. By extrapolating these results, our assay based on Caur_3 primer set would have an analytical LoD of 1.95-2.23 genomes per disk (LoD defined as the extrapolated detection of 3 positive wells per disk on average with a 95% confidence interval of detecting at least one positive well).

TABLE 6
Determination of the analytical limit of detection.

| | | Internal | | | |
|---|---|---|---|---|---|
| | | Control | | | |
| | # | (Avg Cq ± | | Avg Cq ± | |
| Condition | Disks | std dev) | Target | std dev | PPW |
| 5 genomes | 20 | 50 ± 8.5 | Caur_3 | 59 ± 8.6 | 25.6% |
| | | | Detection Rate | 100% (20/20 runs) | |
| 10 genomes | 26 | 49 ± 5.2 | Caur_3 | 51 ± 7.5 | 44.7% |

| | |
|---|---|
| Detection Rate | 100% (26/26 runs) |

Taken together the results described above show that we have designed a LAMP primer set (Caur_3) and optimized a TangenDX™ assay disk that when run in the GeneSpark™ platform is highly specific and sensitive for the detection of C. auris DNA. Caur_3 detects comparably the four C. auris clades (I, II, III & IV), and does not cross-react with any other close Candida spp. and other related yeasts tested. Ten genomes of C. auris in our assay with Caur_3 primer set would result in approximately 15 wells (50%) reporting positive signal. The analytical LoD of Caur_3 primer set is estimated at 1.95-2.23 genomes per disk.

### Description of the Filtration Process and Materials.

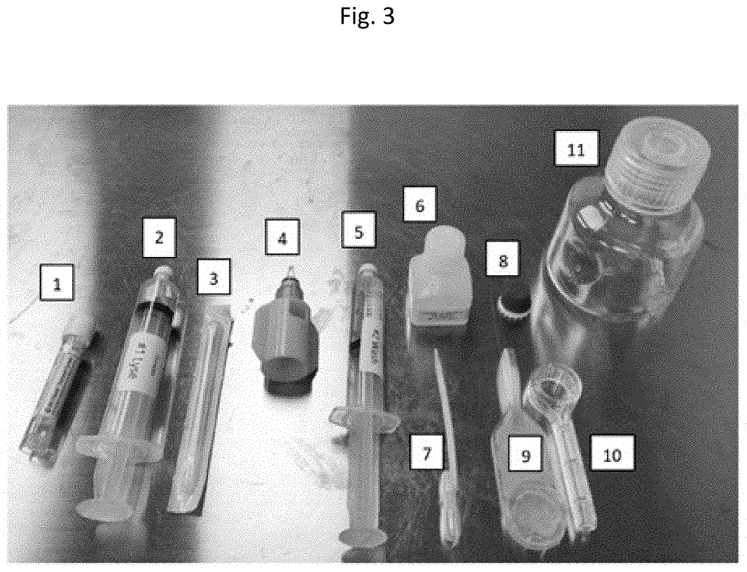

The CDC recommended approach for screening of Candida auris colonization is using a composite swab of the patient's bilateral axilla and groin. These sites, which are the most common and consistent sites of colonization, are generally swabbed with a nylon-flocked swab (BD ESwab collection and transport system), and the swab introduced into a tube containing 1 mL of liquid Amies medium. This medium stabilizes the cells and prevent them from growing or lysing until before being delivered to the reference laboratory for testing. We have optimized a procedure compatible with the GeneSpark™ instrument for Candida auris detection based on a swab collection using 1 mL of liquid Amies. The TangenDx™-Candida auris assay will typically include the materials shown in FIG. 3.

The filtration procedure has an approximate duration of 2½ minutes and consists of the following steps:

1. Twist off the lysis syringe cap and twist the syringe onto the blunt needle.

2. Mix the ESwab transport tube containing patient sample by inverting 5 times.

3. Open the transport tube and draw all the medium into the lysis syringe.

4. Twist off the needle and discard it into an appropriate waste container.

5. Twist the lysis syringe on the LVC-adaptor and place the LVC on the provided waste container.

6. Push the plunger down until all the buffer has passed through.

7. Remove the lysis syringe and discard into an appropriate waste container.

8. Twist the wash syringe on the LCV-adaptor.

9. Push the plunger down until all the buffer has passed through.

10. Twist off the wash syringe, draw 10 mL of air, twist on the LVC-adaptor and push the air through.

11. Remove the wash syringe and the LVC adaptor and discard into an appropriate waste container.

12. Open the LVC cap and transfer 4004 of cap buffer into the cap using the transfer pipette.

13. Tighten the LCV on the buffer cap using the LVC holder and tightening wrench.

14. Place the LCV unit into the GeneSpark™ instrument and press start to begin the run.

### Optimization of the Lysis Buffer Composition.

The optimization process has been carried out using C. parapsilosis cells. Our previous work with Candida species indicates that C. parapsilosis cells are the most resistant to lysis using our system and therefore, they are a good surrogate for C. auris.

Experiment 1. Testing of Amies liquid medium compatibility with standard Tangen lysis buffer (KOH-based). a. Procedure: We spiked 10 CFU of C. parapsilosis in 1 mL of liquid Amies (BD ESwab) and proceeded with the filtration protocol described above using Tangen lysis buffer (KOH-based). b. Results: The LVC unit became clogged making filtration of the lysis-Amies solution very hard with manual pressure. Filtration of the wash buffer was also very hard with manual pressure. Only one of the 4 experimental runs resulted in detection of C. parapsilosis DNA.

Experiment 2. Testing of standard Tangen lysis buffer (KOH-based) directly spiked with C. parapsilosis cells. Procedure: We spiked 10 CFU of C. parapsilosis in a syringe containing 5 mL of Tangen lysis buffer and proceeded with the filtration protocol described above. Results: The lysis and wash buffers could be filtered easily indicating that the source of the clogging was the liquid Amies (likely the agar component). We detected C. parapsilosis DNA in all the runs (13 of 13) with an average percentage of wells being positive (PPW) of 62.1%.

Experiment 3. Testing of Amies liquid medium compatibility with alternative 10 mM citric acid lysis buffer. Procedure: We spiked 10 CFU of C. parapsilosis in 1 mL of liquid Amies (BD ESwab) and proceeded with the filtration protocol described above using 10 mM citric acid lysis buffer. b. Results: The lysis and wash buffer could be filtered easily indicating that citric acid was degrading the agar in liquid Amies. We detected C. parapsilosis DNA in all the runs (5 of 5) with an average PPW of 42.7%. Overall, this indicated that citric acid was compatible with the chemistry of our assay while being able to degrade the agar in liquid Amies.

Experiment 4. Testing if incubation of Amies-citric acid solution increases the performance of the assay. Procedure: We spiked 10 CFU of C. parapsilosis in 1 mL of liquid Amies (BD ESwab) and proceeded with the filtration protocol described above using 10 mM citric acid lysis buffer. After drawing of Amies into the lysis syringe, the solution was vortexed for 30 seconds and incubated at room temperature for 2 minutes. Results: We detected C. parapsilosis DNA in all the runs (8 of 8) with an average percentage of wells being positive (PPW) of 56.7%. Therefore, no significant improvement in PPW was observed with the incubation step.

Experiment 5. Testing if increased concentrations of citric acid of increases the performance of the assay. Procedure: We spiked 10 CFU of C. parapsilosis in 1 mL of liquid Amies (BD ESwab) and proceeded with the filtration protocol described above using 10 mM, 100 mM and 1M citric acid lysis buffer. Results: We detected C. parapsilosis DNA in all the runs (8 of 8) for all the concentrations tested. The PPW was similar between conditions (46%, 40.0% and 50.8%). Therefore, no significant improvement was observed with the increased concentrations tested.

TABLE 7
Results of experiments 1-5 above.

| Condition | # Runs | Detection rate | PPW |
|---|---|---|---|
| Standard Tangen KOH lysis | 4 | 25% (1/4 runs) | 8.3% |
| buffer | | | |
| Direct spike in Tangen KOH | 13 | 100% (13/13 runs) | 62.1% |
| lysis buffer | | | |
| 10 mM citric acid | 5 | 100% (5/5 runs) | 42.7% |
| 10 mM citric acid + incubation | 8 | 100% (8/8 runs) | 56.7% |
| 100 mM citric acid | 8 | 100% (8/8 runs) | 40.0% |
| 1M citric acid | 8 | 100% (8/8 runs) | 50.8% |

Taken together the result of the experiments performed indicate that a lysis buffer consisting in a 10 mM solution of citric acid is compatible with our assay chemistry and is able to liquify the agar in the Amies medium making it easily filterable. In addition, citric acid at this concentration does not represent a potential hazard for the user. Incubation of liquid Amies with citric acid for additional time or increasing the concentration of citric acid does not significantly improve the performance of the assay. We have therefore selected 10 mM citric acid as the final composition of the lysis buffer.

### Determination of Sensitivity, Limit of Detection and Efficiency of Cell Capture

Determination of sensitivity and limit of detection

Procedure: We spiked a range of concentrations (5, 10, 20 and 40 CFU) of C. parapsilosis in 1 mL of liquid Amies (BD ESwab) and proceeded with the filtration protocol described above using 10 mM citric acid lysis buffer. Results: We detected C. parapsilosis DNA in 73.3% of the runs with 5 CFU/mL, 97.4% of the runs with 10 CFU/mL and 100% of the runs at 20 and 50 CFU/mL (Table 8). Therefore, the sensitivity of the assay to detect C. parapsilosis in liquid Amies medium is <10 CFU/mL (defining sensitivity as the detection in 95% of instances). Given the large variability inherent with low concentration cell dilutions, the lower detection at 5 CFU/mL may be related to a lower true input in the system.

TABLE 8
Results of different cell range inputs using filtration system

| Condition | # Runs | Detection rate | PPW |
|---|---|---|---|
| 5 CFU/mL | 15 | 73.3% (11/15 runs) | 26.2% |
| 10 CFU/mL | 38 | 97.4% (37/38 runs) | 54.1% |
| 20 CFU/mL | 7 | 100% (7/7 runs) | 70.0% |
| 50 CFU/mL | 8 | 100% (8/8 runs) | 89.6% |

### Determination of Efficiency of Cell Capture

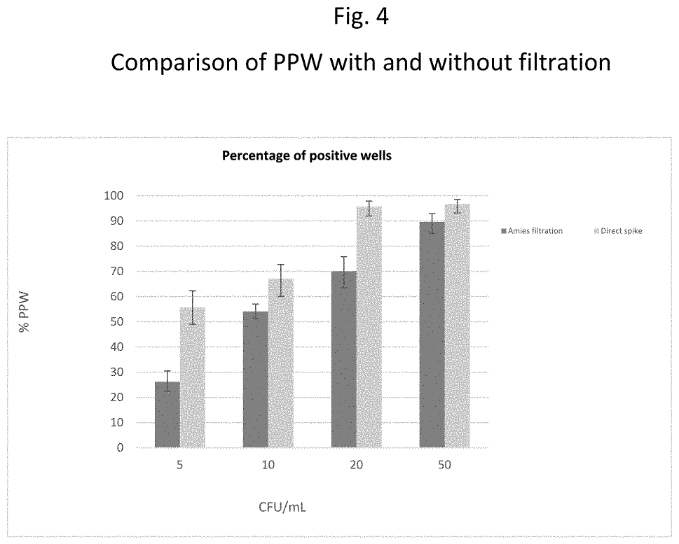

Procedure: We spiked a range of concentrations (5, 10, 20 and 40 CFU) of C. parapsilosis directly into the buffer cap, thus avoiding the filtration system. Results: We detected C. parapsilosis DNA in all the runs at all concentrations (5, 10, 20 and 50 CFU/mL). These are higher detections than those with the filtration device detailed above. The PPW for each of the dilutions was also higher, indicating that some cells were lost during the filtration process (Table 9 and FIG. 4). The estimated efficiency of capture was above 70% for all concentrations, except for 5 CFU/mL for which it was 47.0% (Table 10). This lower efficiency of capture at 5 CFU/mL inputs resulted in the decreased detection rate of 73.3%.

TABLE 9
Results of different cell range inputs without using filtration system

| Condition | # Runs | Detection rate | PPW |
|---|---|---|---|
| 5 CFU/mL | 7 | 100% (7/7 runs) | 55.7% |
| 10 CFU/mL | 7 | 100% (7/7 runs) | 67.1% |
| 20 CFU/mL | 7 | 100% (7/7 runs) | 95.7% |
| 50 CFU/mL | 7 | 100% (7/7 runs) | 96.7% |

TABLE 10
Calculated cell capture efficiency with filtration system

| | Efficiency of |
|---|---|
| Condition | capture |
| 5 CFU/mL | 47.0% |
| 10 CFU/mL | 80.6% |
| 20 CFU/mL | 73.1% |
| 50 CFU/mL | 92.7% |

We have developed a disposable skin-swab based Amies Medium sample process compatible with our assay. With our process we are able to concentrate Candida cells contained in 1 mL of Amies medium in our LVC by using a lysis buffer with 10 mM citric acid. Overall, our filtration procedure, which takes approximately 2½ minutes, concentrates, and detects small amounts of C. parapsilosis cells with a sensitivity below 10 CFU/mL. Given that C. parapsilosis cells are more difficult to lyse than C. auris cells, therefore we expect even higher sensitivity for C. auris detection using our system.

### Example II

This example describes a particular embodiment of the invention directed to the detection of live SARS-CoV-2. The SARS-CoV-2 isolate used for these studies, which is known as USA WA1/2020, was isolated from the first documented US case of a traveler from Wuhan, China. 1 SARS-CoV-2 was sourced from the World Reference Center for Emerging Viruses and Arboviruses (WRCEVA). The SARS-COV-2 isolate was cultured in Vero E6 cells per established procedures. Briefly, 3×106 Vero E6 cells were plated into a T75 flask with 15 mL infection media (Dulbecco's Modified Eagle's medium supplemented with 5% fetal bovine serum and nonessential amino acids) and incubated in a humidified incubator with 5% CO2. The following day the Vero cells were re-fed with infection media and inoculated with 0.5 ml of virus stock. Cells were incubated for 4 days at which point widespread cytopathic effect (CPE) was apparent. At this point, supernatant was collected and 1 mL aliquots of virus stock frozen at −70° C.

For determination of TCID₅₀ an aliquot of virus stock was thawed and TCID₅₀ determined following established procedures. In brief, 10-fold serial dilutions of virus stock were prepared and plated (8 wells per dilution) in a 96 well plate containing 10,000 Vero E6 cells/well. After 5 days of incubation, each well was scored as positive or negative for CPE and TCID₅₀/mL, as determined by the Reed and Muench method. The coronavirus source information and TCID₅₀/mL concentration of the neat virus stock prepared by MRIGlobal is summarized in Table 11.

TABLE 11
Summary of coronaviruses used in the studies

| | | | | Culture | TCID₅₀/ | |
|---|---|---|---|---|---|---|
| Virus | Isolate | Source/No. | Lot No. | Date | ml | GC/ml |
| SARS- | USA | WRCEVA | TVP23156 | Jun. 1, | 1.95 × | 3.3 × |
| CoV-2 | WA1/ | | | 2020 | 106 | 109 |
| | 2020 | | | | | |

GC/ml = Genomic Copies/ml.

Quantitative RT-PCR of SARS-CoV-2 Stock Using N1 Primers and Probes (CDC Method). Viral genomic copies per mL (GC/mL) was determined by quantitative RT-PCR using a Bio-Rad CFX96 Real-Time Detection System. The standard curve was prepared from a custom gBlock (Integrated DNA Technologies, San Diego Calif.) containing the entire SARS-CoV-2 N1 target amplicon sequence plus 30 by of flanking sequence on the 5′ and 3′ ends. The gBlock sequence was derived from NCBI accession number MN908947 (Severe acute respiratory syndrome coronavirus 2 isolate Wuhan-Hu-I, complete genome). The copy number concentration of the gBlock was determined based on the total amount of oligonucleotide (ng) and the length (b).

The RT-qPCR. procedure used the 2019-nCoV RUO Kit from Integrated DNA Technologies, which includes assays for N1, N2 and Rp with premixed primers and probes. TaqPath™ 1-step RT-qPCR Master Mix, CG was sourced from ThermoFisher™. Thermal cycling conditions followed those published in the CDC 2019-Novel Coronavirus (2019-nCoV) Real-Time RT-PCR Diagnostic Panel Instructions for Use and are summarized in Table 12.

TABLE 12
CDC assay thermal cycling parameters

| Stage | Temperature | Time | | Cycles |
|---|---|---|---|---|
| 1 | 25° C. | 2 | min | 1 |
| 2 | 50° C. | 15 | min | 1 |
| 3 | 95° C. | 2 | min | 1 |
| 4 | 95° C. | 3 | sec | 45 |
| | 55° C. | 30 | sec | |

The gBlock standard curve consisted of the following concentrations: 1×101, 1×102, 1×103, and 1×104 GC/μL. SARS-CoV-2 culture supernatant was diluted in nuclease-free water for testing at the following dilutions: 10-1, 1-0 2, 10-3, 104, 1-0_5. Master mix was prepared as shown in Table 13.

TABLE 13
CDC assay master mix preparation

| | Volume per |
|---|---|
| Reagent | Reaction |
| Nuclease-free water | 8.5 μL |
| 2019-nCoV RUO Kit | 1.5 μL |
| TaqPath ™ 1-step RT-qPCR Master Mix | 5.0 μL |
| TOTAL | 15 μL |

For the RT-PCR reaction, 15 μL of prepared master mix was added to each well followed by 5 μL of standard or sample, for a final total volume of 20 μL per reaction well. Both gBlock standards and SARS-CoV-2 sample dilutions were run in duplicate wells. The GC/mL of the SARS-CoV-2 dilutions was determined using the slope and y-intercept of the gBlock standard curve, as determined by linear regression analysis. The GC/mL of the virus stock was determined based on the average of the duplicate well results for all dilutions tested. For the SARS-COV-2 stock used for these studies, the concentration was calculated to be 3.3×109

### Limits of Detection.

Range finding. A preliminary LoD was determined by first testing a range of dilutions (120, 60, 30, 15, 4.5 and 0 copies/0) of SARS-CoV-2 Working Stock #5, diluted in simulated nasal matrix (Table 14). The simulated nasal matrix was made by eluting two negative donor nasopharyngeal swabs in 10 mL of Tangen Assay Buffer v.5 (TAB). A 50 μL sample of SARS-CoV-2 diluted in SNM was added to a fresh, sterile NP swab, then eluted in a fresh, unused vial of 5 mL Tangen Assay Buffer.

TABLE 14
SARS-COV-2 Working Stock Dilution - LOD Range Finding

| | |
|---|---|
| SARS-COV-2 | 3.3e9 GC/ml; |
| Working Stock | 3.3e6 GC/μL |

| Starting | Target | | | |
|---|---|---|---|---|
| Conc. | Cone. | Target | Stock | |
| (copies/ | (copies/ | Vol | Vol | Diluent Vol |
| μL) | μL) | (μL) | (μL) | (μL) |
| 3.30E+06 | 1.00E+05 | 800 | 24.24 | 775.76 |
| 1.00E+05 | 5.00E+03 | 800 | 40.00 | 760.00 |
| 5.00E+03 | 2.50E+03 | 800 | 400.00 | 400.00 |

| | | | | Vol. of | | |
|---|---|---|---|---|---|---|
| | | | Vol | Tangen | | |
| | Final | | of | Sample | Copies | Copies |
| Viral | Cone. | | Viral | Buffer | per | per |
| Stock | (GC/ | Total | Stock | with | swab | ml |
| Cone. | μL) | Vol.* | (μL) | NP (μL) | (50 μ) | TAB |
| 5,000 | 120 | 1500 | 36.0 | 1,464 | 6000 | 1200 |
| | 60 | 1500 | 18.0 | 1,482 | 3000 | 600 |
| | 30 | 1500 | 9.0 | 1,491 | 1500 | 300 |
| 2,500 | 15 | 1500 | 9.0 | 1,491 | 750 | 150 |
| | 4.5 | 1500 | 2.7 | 1,497.3 | 225 | 45 |

The eluted, spiked TAB samples were then processed according to the Tangen x SARS-CoV-2 Assay as described in the TangenDx SARS-Cov-2 Assay Instructions for. Each virus dilution was tested in triplicate. We successfully detected SARS-COV-2 in 3 of 3 replicates at 4.5 copies/μL or 45 copies per mL of TAB added to the Tangen SANS-COV-2 Molecular Test. The results of the preliminary LoD testing are summarized in Table 15, and component information is listed in Table 16.

TABLE 15
Preliminary Limit of Detection (LoD) Determination

| | Test Results | | |
|---|---|---|---|
| | Replicate | Replicate | Replicate |
| Sample | 1 | 2 | 3 |
| 120 copies/μL | + | + | + |
| 60 copies/μL | + | + | + |
| 30 copies/μL | + | + | + |
| 15 copies/μL | + | + | + |
| 4.5 copies/μL | + | + | + |
| 0 copies/μL | − | − | − |

TABLE 16
Components used for LoD Determination

| | | Part | Lot |
|---|---|---|---|
| Component | Vendor/Manufacturer | No. | No. |
| Covid assay Disk | Tangen Biosciences | KRW | 204041-01 |
| | | 0165 | |
| Tangen Assay Buffer | Tangen Biosciences | KRW | 20A42001 |
| v.5 | | 0178 | |
| 1.5 ml Microfuge Tubes | Costar | 3213 | 04920000 |
| LVC Caps with Beads | Tangen Biosciences | KRW | CoV-2-EVL- |
| | | 0185 | 20-200-826 |
| Positive Control | Tangen Biosciences | KRW | CoV-2-EVL- |
| Beads | | 0179 | 200-720-PC5 |
| Negative Control | Tangen Biosciences | KRW | CoV-2-NC-EVL- |
| Beads | | 0181 | 20200-728 |
| Sample Collection | Tangen Biosciences | KRR0 | TBl-20C36004 |
| Swabs | | 195 | |
| 400 μI Bulb Pipette | Tangen Biosciences | KRR0 | TBl-20C42006 |
| | | 225 | |

Simultaneously, samples were prepared for testing with the CDC EUA RT-qPCR assay reference method. In brief, for each dilution point, sterile NP swabs were spiked with 50 μL of SARS-COV-2 viral dilution and then eluted in 1 mL of viral transport media (VTM). For each dilution point, 140 μL of the 1 mL volume was extracted in triplicate using the Qiagen QIAamp DSP Viral RNA Mini kit., with a final elution volume of 140 μL.

TABLE 17
SARS-COV-2 Working Stock Dilution - LoD Range Finding

| | | | Vol. | Vol. of | | | |
|---|---|---|---|---|---|---|---|
| | Final | | of | Tangen | Copies | Copies | |
| Viral | Cone. | | Viral | Sample | per | per | Copies |
| Stock | (GC/ | Total | Stock | Buffer | swab | mL | per |
| Cone. | μL) | Vol. | (μL) | with NP | (50 μL) | VTM | 140 ul |
| 5,000 | 120 | 1500 | 36.0 | 1,464 | 6000 | 6000 | 840 |
| | 60 | 1500 | 18.0 | 1,482 | 3000 | 3000 | 420 |
| | 30 | 1500 | 9.0 | 1,491 | 1500 | 1500 | 210 |
| 2,500 | 15 | 1500 | 9.0 | 1,491 | 750 | 750 | 105 |
| | 4.5 | 1500 | 2.7 | 1,497.3 | 225 | 225 | 31.5 |

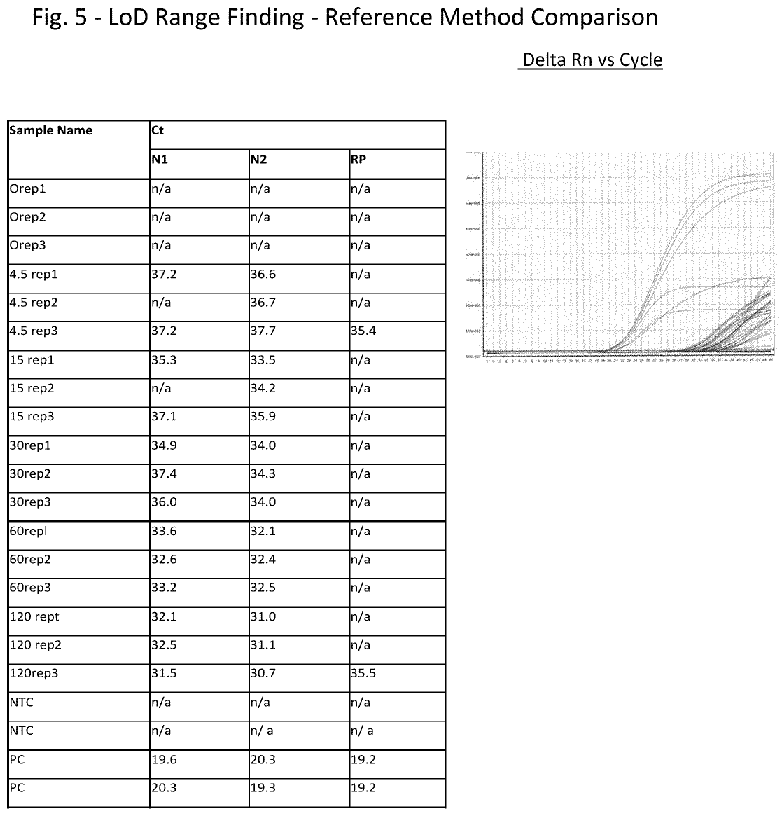

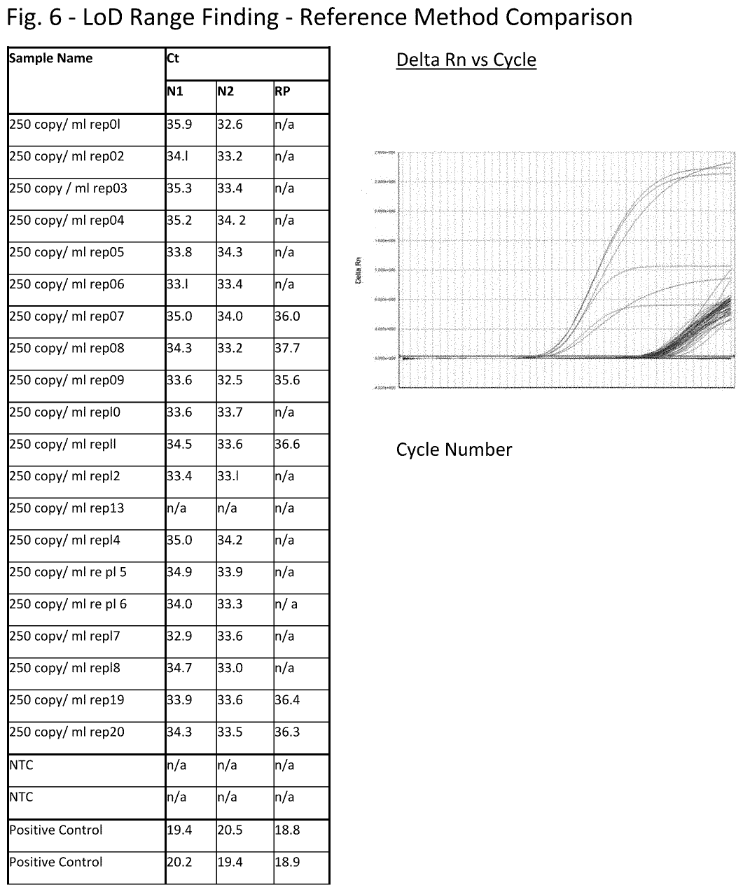

Extracted nucleic acids were tested in single replicates with the CDC EUA RT-qPCR assay, using the Integrated DNA Technologies 2019-nCoV RUO Kit for N1, N2 and RP assays. We used the assay parameters as provided in the FDA CDC 2019-No ref Coronavirus (2019-nCoV) Real-Time RT-PCR Diagnostic Panel Instructions for Use. With the reference method, we detected SARS-COV-2 with both N1 and N2 assays at 30 copies/AL. At 15 and 4.5 copies per μL, we observed inconsistent detection with N1 and N2. We did not expect consistent detection with the RP assay since the contrived samples were prepared without nasal matrix background. The results of the reference method testing are summarized in FIG. 5. We also performed the reference method for LoD confirmation as described in Tangen Protocol TAN-004-NCLN02. We spiked 50 μL of the same diluted SARS-COV-2 virus (25 copies/μL) on sterile NP swabs and elated into 1 mL of VTM (Table 17). Using the same reference method protocol as described above, we processed twenty replicate samples with the CDC EUA RT-qPCR assay. We observed detection of SARS-COV-2 with both N1 and N2 with 19 of 20 replicates. The results of the reference method testing arc summarized in FIG. 6.

The Limit of Detection for the Tangen-Dx SARS CoV-2 Molecular Test was confirmed to be 250 viral genomic copies per mL. This confirmation was performed alongside the CDC EUA RT-qPCR assay as a reference method, which showed similar performance.

Other aspects of the invention may be described in the follow exemplary embodiments:

1. A method of detecting a nucleic acid of one or more microorganism in a subject, the method comprising, independent of order, the following steps:
a. obtaining an upper respiratory sample from a subject;
b. processing the upper respiratory sample in an apparatus to capture and lyse microorganisms from the sample, and obtaining a nucleic acid extract from microorganisms in the upper respiratory sample of a subject;
c. selecting one or more target sequence from a microorganism of interest, and selecting one or more nucleic acid amplification primer set that is complementary to at least a portion of a target sequence from a microorganism of interest;
d. incubating the target sequence with the one or more nucleic acid amplification primer set in a reaction mixture and performing an amplification reaction; and
e. detecting one or more target sequence from a microorganism of interest.

2. A method according to embodiment 1, wherein the incubation step includes a pre-amplification step before step d) that uses random primers and reagents for the nonselective amplification of nucleic acid from microorganisms in the sample to produce a pre-amplification product.

3. A method according to embodiment 1, wherein an upper respiratory sample from a subject comprised samples from a nasal pharyngeal swab, a nasal swab, a throat swab, saliva, a nasal aspirate, and any other method suitable to obtain sufficient sample.

4. A method according to embodiment 1, wherein more than one microorganism in a subject's upper respiratory sample can be detected.

5. A method according to embodiment 1, wherein the microorganism comprises a virus.

6. A method according to embodiment 4, wherein the virus comprises a coronavirus

7. A method according to embodiment 4, wherein the virus is a SARS-CoV-2 type virus.

8. A method according to embodiment 4, wherein the virus is selected from Adenovirus, Coronavirus HKU1, Coronavirus NL63, Coronavirus 229E, Coronavirus OC43, Human Metapneumovirus, Human Rhinovirus/Enterovirus, Influenza A, Influenza B, Parainfluenza Virus 1, Parainfluenza Virus 2, Parainfluenza Virus 3, Parainfluenza Virus 4, Respiratory Syncytial Virus, and SARS-CoV-2 type virus.

9. A method according to embodiment 1, wherein the amplified template is detected or quantified in real time.

10. A method according to embodiment 1, wherein the amplification is isothermal.

11. A method according to embodiment 1, wherein the target sequence comprises a SARS-CoV-2 type virus nucleic acid sequence in the nucleocapsid recombinant N2 fragment domain or in the nucleocapsid recombinant N3 fragment domain.

12. A method according to embodiment 1, wherein the target sequence comprises a SARS-CoV-2 type virus nucleic acid sequence in the nucleocapsid recombinant N2 fragment domain the target sequence comprises a nucleocapsid recombinant N3 fragment domain.

13. A method according to embodiment 1, wherein the target sequence comprises a SARS-CoV-2 type virus nucleic acid sequence and the primers that are complementary to at least a portion of that target sequence are selected from CTGAGGGAGCCTTGAATACACCAA (SEQ ID NO:1); CGCCATTGCCAGCCATTCTAGC (SEQ ID NO:2); TCCCTTCTGCGTAGAAGCCTTTTGGC-CCCGCAATCCTGCTAACAATGCT (SEQ ID NO:3); CAGAGGCGGCAGTCAAGCCTCTTC-CCCCTACTGCTGCCTGGAGTT (SEQ ID NO:4); GTTGTTCCTTGAGGAAGTTGTAGCACGA (SEQ ID NO:5); CGTTCCTCATCACGTAGTCGCAACAG (SEQ ID NO:6); ATGGAGAACGCAGTGGGGC (SEQ ID NO:7); TCATTTTACCGTCACCACCACGAA (SEQ ID NO:8); GCCATGTTGAGTGAGAGCGGTGAACC-GCGATCAAAACAACGTCGGCC (SEQ ID NO:9); AATTCCCTCGAGGACAAGGCGTTCCA-TGGTAGCTCTTCGGTAGTAGCCAA (SEQ ID NO:10); AGACGCAGTATTATTGGGTAAACCTTGG (SEQ ID NO:11); and ATTAACACCAATAGCAGTCCAGATGACCA (SEQ ID NO:12).

14. A method according to embodiment 1, wherein the target sequence comprises a C. auris nucleic acid sequence and the primers that are complementary to at least a portion of that target sequence are selected from CGGCGAGTTGTAGTCTGGA (SEQ ID NO:13); TCCATCACTGTACTTGTTCGCT (SEQ ID NO:14); GGGCCACAGGAAGCACTAGCACAGCAGGCAAGTCCTTTGG (SEQ ID NO:15); CCGACGAGTCGAGTTGTTTGGGCGGTCTCTCGCCAATATTTAGC (SEQ ID NO:16); AAAGCAGGTACGGGGCTG (SEQ ID NO:17); and GCAGCTCTAAGTGGGTGGTA (SEQ ID NO:18).

15. A kit for detecting or quantifying a target nucleic acid in a nucleic acid sample, the kit comprising a solid phase disc for detecting nucleic acids comprising one or more amplification primer sets and one or more second primer sets; and ii) instructions for use of the disk for a method of detecting a microorganism in a nucleic acid sample from a subject on an apparatus, instrument, or system described herein.

16. A method of detecting a nucleic acid of one or more microorganism in a subject, the method comprising, independent of order, the following steps:
a) obtaining a blood or blood fraction sample from a subject;
b) processing the blood sample in an apparatus to capture and lyse microorganisms from the sample, and obtaining a nucleic acid extract from microorganisms in the blood sample of a subject;
c) selecting one or more target sequence from a microorganism of interest, and selecting one or more nucleic acid amplification primer set that is complementary to at least a portion of a target sequence from a microorganism of interest;
d) incubating the target sequence with the one or more nucleic acid amplification primer set in a reaction mixture and performing an amplification reaction; and
e) detecting one or more target sequence from a microorganism of interest.

17. A method according to embodiment 17, wherein the incubation step includes a pre-amplification step before step d) that uses random primers and reagents for the nonselective amplification of nucleic acid from microorganisms in the sample to produce a pre-amplification product.

18. A method according to embodiment 17, wherein more than one microorganism in a subject's blood sample can be detected.

19. A method according to embodiment 17, wherein the microorganism comprises one or more bacteria species.

20. A method according to embodiment 20, wherein the one or more bacteria species is selected from Bordetella parapertussis, Bordetella pertussis, Chlamydia pneumoniae, Mycoplasma pneumoniae, Escherichia Coli, Klebsiella pneumoniae, Klebsiella oxytoca, Salmonella, Proteus mirabilis, Citrobacter freundii, Serratia marcescens, Enterococcus faecalis, Enterococcus faecium, Staphylococcus aureus, Staphylococcus epidermidis, Staphylococcus lugdunensis, and Streptococcus pneumoniae.

21. A method according to embodiment 20, wherein one or more bacterium species is a pathogenic bacterium.

22. A method according to embodiment 22, wherein the bacterium B. anthracis is detected.

23. A method according to embodiment 23, wherein the target nucleic acids comprise pXO1 and pXO2 nucleic acid sequences from bacterium B. anthracis.

24. A method according to embodiment 17, wherein a target sequence comprises nucleic acids from genes that confer antimicrobial resistance (AMR) to bacteria.

25. A method according to embodiment 17, wherein one or more antimicrobial resistance gene (AMR) is detected from one or more bacterial pathogen species suspected of being present in a subject's blood sample.

26. A method according to embodiment 17, wherein at least 10 species of bacteria and their corresponding antibiotic resistance genes are analyzed.

27. A method according to embodiment 17, wherein between about 10 and about 20 species of bacteria and their corresponding antibiotic resistance genes are analyzed.

28. A method according to embodiment 17, wherein the amplified template is detected or quantified in real time.

29. A method according to embodiment 17, wherein the amplification is isothermal.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the embodiments. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the embodiments. It is also that as used herein and in the appended embodiments, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended embodiments.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following embodiments. In addition, where features or aspects of the invention are described in terms of Markush groups, those skilled in the art will recognize that the invention is also thereby described in terms of any individual member or subgroup of members of the Markush group.

What is claimed is:

1. A method of detecting a nucleic acid of one or more microorganism in a subject, the method comprising, independent of order, the following steps:
a. obtaining an upper respiratory sample from a subject;
b. processing the upper respiratory sample in an apparatus to capture and lyse microorganisms from the sample, and obtaining a nucleic acid extract from microorganisms in the upper respiratory sample of a subject;
c. selecting one or more target sequence from a microorganism of interest, and selecting one or more nucleic acid amplification primer set that is complementary to at least a portion of a target sequence from a microorganism of interest;
d. incubating the target sequence with the one or more nucleic acid amplification primer set in a reaction mixture and performing an amplification reaction; and
e. detecting one or more target sequence from a microorganism of interest.

2. A method according to embodiment 1, wherein the incubation step includes a pre-amplification step before step d) that uses random primers and reagents for the nonselective amplification of nucleic acid from microorganisms in the sample to produce a pre-amplification product.

3. A method according to embodiment 1, wherein an upper respiratory sample from a subject comprised samples from a nasal pharyngeal swab, a nasal swab, a throat swab, saliva, a nasal aspirate, and any other method suitable to obtain sufficient sample.

4. A method according to embodiment 1, wherein more than one microorganism in a subject's upper respiratory sample can be detected.

5. A method according to embodiment 1, wherein the microorganism comprises a virus.

6. A method according to embodiment 4, wherein the virus comprises a coronavirus

7. A method according to embodiment 4, wherein the virus is a SARS-CoV-2 type virus.

8. A method according to embodiment 4, wherein the virus is selected from Adenovirus, Coronavirus HKU1, Coronavirus NL63, Coronavirus 229E, Coronavirus OC43, Human Metapneumovirus, Human Rhinovirus/Enterovirus, Influenza A, Influenza B, Parainfluenza Virus 1, Parainfluenza Virus 2, Parainfluenza Virus 3, Parainfluenza Virus 4Respiratory Syncytial Virus, and SARS-CoV-2 type virus.

9. A method according to embodiment 1, wherein the amplified template is detected or quantified in real time.

10. A method according to embodiment 1, wherein the amplification is isothermal.

11. A method according to embodiment 1, wherein the target sequence comprises a SARS-CoV-2 type virus nucleic acid sequence in the nucleocapsid recombinant N2 fragment domain or in the nucleocapsid recombinant N3 fragment domain.

12. A method according to embodiment 1, wherein the target sequence comprises a SARS-CoV-2 type virus nucleic acid sequence in the nucleocapsid recombinant N2 fragment domain the target sequence comprises a nucleocapsid recombinant N3 fragment domain.

13. A method according to embodiment 1, wherein the target sequence comprises a SARS-CoV-2 type virus nucleic acid sequence and the primers that are complementary to at least a portion of that target sequence are selected from CTGAGGGAGCCTTGAATACACCAA (SEQ ID NO:1); CGCCATTGCCAGCCATTCTAGC (SEQ ID NO:2); TCCCTTCTGCGTAGAAGCCTTTTGGC-CCCGCAATCCTGCTAACAATGCT (SEQ ID NO:3); CAGAGGCGGCAGTCAAGCCTCTTC-CCCCTACTGCTGCCTGGAGTT (SEQ ID NO:4); GTTGTTCCTTGAGGAAGTTGTAGCACGA (SEQ ID NO:5); CGTTCCTCATCACGTAGTCGCAACAG (SEQ ID NO:6); ATGGAGAACGCAGTGGGGC (SEQ ID NO:7); TCATTTTACCGTCACCACCACGAA (SEQ ID NO:8); GCCATGTTGAGTGAGAGCGGTGAACC-GCGATCAAAACAACGTCGGCC (SEQ ID NO:9); AATTCCCTCGAGGACAAGGCGTTCCA-TGGTAGCTCTTCGGTAGTAGCCAA (SEQ ID NO:10); AGACGCAGTATTATTGGGTAAACCTTGG (SEQ ID NO:11); and ATTAACACCAATAGCAGTCCAGATGACCA (SEQ ID NO:12).

14. A method according to embodiment 1, wherein the target sequence comprises a C. auris nucleic acid sequence and the primers that are complementary to at least a portion of that target sequence are selected from CGGCGAGTTGTAGTCTGGA (SEQ ID NO:13); TCCATCACTGTACTTGTTCGCT (SEQ ID NO:14); GGGCCACAGGAAGCACTAGCACAGCAGGCAAGTCCTTTGG (SEQ ID NO:15); CCGACGAGTCGAGTTGTTTGGGCGGTCTCTCGCCAATATTTAGC (SEQ ID NO:16); AAAGCAGGTACGGGGCTG (SEQ ID NO:17); and GCAGCTCTAAGTGGGTGGTA (SEQ ID NO:18).

15. A kit for detecting or quantifying a target nucleic acid in a nucleic acid sample, the kit comprising a solid phase disc for detecting nucleic acids comprising one or more amplification primer sets and one or more second primer sets; and ii) instructions for use of the disk for a method of detecting a microorganism in a nucleic acid sample from a subject on an apparatus, instrument, or system described herein.

16. A method of detecting a nucleic acid of one or more microorganism in a subject, the method comprising, independent of order, the following steps:
a) obtaining a blood or blood fraction sample from a subject;
b) processing the blood sample in an apparatus to capture and lyse microorganisms from the sample, and obtaining a nucleic acid extract from microorganisms in the blood sample of a subject;
c) selecting one or more target sequence from a microorganism of interest, and selecting one or more nucleic acid amplification primer set that is complementary to at least a portion of a target sequence from a microorganism of interest;
d) incubating the target sequence with the one or more nucleic acid amplification primer set in a reaction mixture and performing an amplification reaction; and
e) detecting one or more target sequence from a microorganism of interest.

17. A method according to embodiment 17, wherein the incubation step includes a pre-amplification step before step d) that uses random primers and reagents for the nonselective amplification of nucleic acid from microorganisms in the sample to produce a pre-amplification product.

18. A method according to embodiment 17, wherein more than one microorganism in a subject's blood sample can be detected.

19. A method according to embodiment 17, wherein the microorganism comprises one or more bacteria species.

20. A method according to embodiment 20, wherein the one or more bacteria species is selected from Bordetella parapertussis, Bordetella pertussis, Chlamydia pneumoniae, Mycoplasma pneumoniae, Escherichia Coli, Klebsiella pneumoniae, Klebsiella oxytoca, Salmonella, Proteus mirabilis, Citrobacter freundii, Serratia marcescens, Enterococcus faecalis, Enterococcus faecium, Staphylococcus aureus, Staphylococcus epidermidis, Staphylococcus lugdunensis, and Streptococcus pneumoniae.


SEQUENCE LISTING (18 sequences)

SEQ ID NO: 1 - DNA, 24 bp, Artificial Sequence
  Description of Artificial Sequence Synthetic primer
ctgagggagccttgaatacaccaa

SEQ ID NO: 2 - DNA, 22 bp, Artificial Sequence
  Description of Artificial Sequence Synthetic primer
cgccattgccagccattctagc

SEQ ID NO: 3 - DNA, 49 bp, Artificial Sequence
  Description of Artificial Sequence Synthetic primer
tcccttctgcgtagaagccttttggccccgcaatcctgctaacaatgct

SEQ ID NO: 4 - DNA, 45 bp, Artificial Sequence
  Description of Artificial Sequence Synthetic primer
cagaggcggcagtcaagcctcttccccctactgctgcctggagtt

SEQ ID NO: 5 - DNA, 28 bp, Artificial Sequence
  Description of Artificial Sequence Synthetic primer
gttgttccttgaggaagttgtagcacga

SEQ ID NO: 6 - DNA, 26 bp, Artificial Sequence
  Description of Artificial Sequence Synthetic primer
cgttcctcatcacgtagtcgcaacag

SEQ ID NO: 7 - DNA, 19 bp, Artificial Sequence
  Description of Artificial Sequence Synthetic primer
atggagaacgcagtggggc

SEQ ID NO: 8 - DNA, 24 bp, Artificial Sequence
  Description of Artificial Sequence Synthetic primer
tcattttaccgtcaccaccacgaa

SEQ ID NO: 9 - DNA, 47 bp, Artificial Sequence
  Description of Artificial Sequence Synthetic primer
gccatgttgagtgagagcggtgaaccgcgatcaaaacaacgtcggcc

SEQ ID NO: 10 - DNA, 50 bp, Artificial Sequence
  Description of Artificial Sequence Synthetic primer
aattccctcgaggacaaggcgttccatggtagctcttcggtagtagccaa

SEQ ID NO: 11 - DNA, 28 bp, Artificial Sequence
  Description of Artificial Sequence Synthetic primer
agacgcagtattattgggtaaaccttgg

SEQ ID NO: 12 - DNA, 29 bp, Artificial Sequence
  Description of Artificial Sequence Synthetic primer
attaacaccaatagcagtccagatgacca

SEQ ID NO: 13 - DNA, 19 bp, Artificial Sequence
  Description of Artificial Sequence Synthetic primer
cggcgagttgtagtctgga

SEQ ID NO: 14 - DNA, 22 bp, Artificial Sequence
  Description of Artificial Sequence Synthetic primer
tccatcactgtacttgttcgct

SEQ ID NO: 15 - DNA, 40 bp, Artificial Sequence
  Description of Artificial Sequence Synthetic primer
gggccacaggaagcactagcacagcaggcaagtcctttgg

SEQ ID NO: 16 - DNA, 44 bp, Artificial Sequence
  Description of Artificial Sequence Synthetic primer
ccgacgagtcgagttgtttgggcggtctctcgccaatatttagc

SEQ ID NO: 17 - DNA, 18 bp, Artificial Sequence
  Description of Artificial Sequence Synthetic primer
aaagcaggtacggggctg

SEQ ID NO: 18 - DNA, 20 bp, Artificial Sequence
  Description of Artificial Sequence Synthetic primer
gcagctctaagtgggtggta